(19) United States
(12) Patent Application Publication
(10) Pub. No.: US 2026/0234194 A1
(43) Pub. Date: Aug. 13, 2026

(54) OCCIDIOFUNGIN ANALOGS AND METHODS OF MAKING AND USING

(71) Applicants: THE TEXAS A&M UNIVERSITY SYSTEM, COLLEGE STATION, TX (US); SANO CHEMICALS, BRYAN, TX (US)

(72) Inventors: JAMES LEIF SMITH, BRYAN, TX (US); NOPAKORN HANSANANT, COLLEGE STATION, TX (US); RAVI ORUGUNTY, ROUND ROCK, TX (US)

(21) Appl. No.: 19/152,989

(22) PCT Filed: Feb. 2, 2024
(86) PCT No.: PCT/US2024/014175

§ 371 Date: Aug. 1, 2025

Related U.S. Application Data
(60) Provisional application No. 63/443,060, filed on Feb. 3, 2023.

(51) Int. Cl. C07K 9/00; A61K 38/00; A61P 31/10

(52) U.S. Cl. CPC C07K 9/006; A61P 31/10; A61K 38/00

(57) ABSTRACT

The subject invention pertains to methods of synthesizing occidiofungin analogs using a variant of occidiofungin which contains an aspartic acid (ASP7) or glutamic acid at position 7 (GLU7) instead of the previously characterized asparagine at position 7. Selective semi-synthetic analogs produced using the ASP7 and GLU7 variants displayed improved antifungal activity compared to ASN7 occidiofungin variants. The ASP7 and GLU7 variants of occidiofungin and compositions thereof can be used for treating fungal and parasitic infections as well as cancer.

### CROSS-REFERENCE TO RELATED APPLICATION

This application claims the benefit of U.S. Provisional Application Ser. No. 63/443,060, filed Feb. 3, 2023, the disclosure of which is hereby incorporated by reference in its entirety, including all figures, tables and amino acid or nucleic acid sequences.

### STATEMENT REGARDING FEDERALLY SPONSORED RESEARCH

This invention was made with government support under 2R42AI131792-02A1 awarded by National Institutes of Health. The government has certain rights in the invention.

### STATEMENT REGARDING SEQUENCE LISTING

The Sequence Listing for this application is labeled “Seq-List.xml” which was created on Feb. 1, 2024 and is 32,956 bytes. The entire content of the sequence listing is incorporated herein by reference in its entirety.

### BACKGROUND OF THE INVENTION

Occidiofungin is an antifungal produced by the soil bacterium Burkholderia contaminans MS141. Occidiofungin is a cyclic glycopeptide produced non-ribosomally and is composed of 8 amino acids (FIG. 1).

The proposed mechanism of action of occidiofungin is the disruption of the physiological bioactivity of actin. The fungicidal activity of occidiofungin has been demonstrated in a wide variety of fungal species, including multi-drug resistant C. glabrata and C. auris². These results have led to the development of occidiofungin as an antifungal agent for the treatment of vulvovaginal (demonstrated in a mouse model^{2,3}), oral, and cutaneous fungal infections. Occidiofungin did not show efficacy in in vivo treatment of systemic fungal infections, possibly due to strong binding of the compound to serum proteins in animal model systems³.

Studies have shown that occidiofungin also demonstrates chemotherapeutic activity against several cancer cell lines. The anti-cancer properties were with greater potency compared to its antifungal activity⁴, with minimal toxicity. Occidiofungin has also demonstrated in vitro antiparasitic activity against the gastrointestinal tract parasite Cryptosporidium parvum⁵. Therefore, there remains a need to produce analogs of occidiofungin to provide novel variants that are optimized for treatment of systemic fungal infections, as an anticancer agent, or as an antiparasitic agent.

### BRIEF SUMMARY OF THE INVENTION

The invention provides methods for producing novel occidiofungin analogs that are derived from occidiofungin variants, preferably occidiofungin aspartic acid 7 variants (OCF-I and OCF-J) and glutamic acid 7 variants (OCF-M and OCF-N). The OCF-I and OCF-J variants have an aspartic acid at position 7 (ASP7), and OCF-M and OCF-N have a glutamic acid at position 7 (GLU7), instead of asparagine (ASN7) found in previously isolated and identified occidiofungin variants (OCF-A and OCF-B). In certain embodiments, the occidiofungin variants can be modified by selective modification of the carboxylic acid functional group at position 7 via carbodiimide mediated amidation to produce semi-synthetic analogs.

In certain embodiments, various OCF-I, OCF-J, OCF-M, and OCF-N analogs described possess antifungal activity. In certain embodiments, occidiofungin variants derived from OCF-I, OCF-J, OCF-M, and OCF-N displayed increased antifungal activity compared to the ASP7 and GLU7 variants (e.g., OCF-I, OCF-J, OCF-M, and OCF-N). Accordingly, certain embodiments of the invention provide methods of controlling fungal and parasitic infections by administering to the subjects in need thereof the OCF-I, OCF-J, OCF-M, and OCF-N analogs described herein.

In certain embodiments, the ASP7 or GLU7 residue of the OCF-I, OCF-J, OCF-M, and OCF-N analogs can be modified to contain a linker conjugated to a monoclonal antibody enabling a targeting-based delivery system for specific cancer cells. In certain embodiments, the engineered conjugates to the ASP7 or GLU7 position can contain a variety of additional selective functional groups, such as, for example, an amine, aldehyde, alkyne, or alcohol.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIGS. 1A-1C. Chemical structure of occidiofungin. FIG. 1A) The chemical structure of OCF-A, OCF-B, OCF-I, and OCF-J with numbers denoting the position of the amino acids. FIG. 1B) The chemical structure of OCF-K, OCF-L, OCF-M and OCF-N with numbers denoting the position of the amino acids. FIG. 1C) Representation of the amino acid arrangement. OCF-A and OCF-B have an asparagine residue at position 7. OCF-A (1200.59 Da) has an asparagine residue at position 1 and OCF-B (1216.59 Da) has a β-hydroxy asparagine at position 1. The two variants are not separable by HPLC. OCF-I and OCF-J has an aspartic acid residue at position 7, and a hydrogen or hydroxy group (R1) at position 1 respectively. OCF-K and OCF-L have a glutamine residue at position 7. OCF-K has an asparagine residue at position 1 and OCF-L (1230.61 Da) has a β-hydroxy asparagine at position 1. OCF-M and OCF-N has a glutamic acid residue at position 7. OCF-M has an asparagine residue at position 1 and OCF-N (1231.59 Da) has a β-hydroxy asparagine at position 1 respectively.

FIG. 2. Chromatogram of B. contaminans MS14 extract with acetonitrile (ACN) and 10 mM triethylamine both with 0.1% formic acid (FA) as mobile phases. The percentage of 10 mM Triethylamine 0.1% FA mobile phase starts at 90% and goes down to 20%, with a 10-minute hold at 60%. The sample is injected at 5 minutes into the run, as indicated by the sharp drop in pressure at that point. Absorbance was monitored at 220 nm (amino acids) and 280 nm (aromatic amino acids) for occidiofungin. Occidiofungin has a higher absorbance at 220 nm compared to 280 nm as β-hydroxy tyrosine 4 is the only aromatic amino acid. Table 1 indicates the variants eluting out at the demarcated peaks.

FIGS. 3A-3B. NMR data showing FIG. 3A) The TOCSY fingerprint region of a mixture of OCF-A/OCF-B and OCF-I/OCF-J. The newly assigned ASP7 spin system is shown, and a new spin system assigned as GLN7 is also shown. Other amino acids present in occidiofungin are also labelled. Orange lines indicate unknown spin systems. FIG. 3B) NOESY data showing the NOEs indicating interaction from Hα of GLY6 to the HN of the ASP7 and ASN7 spin system. NOEs not in this region are also seen which indicate interaction of Hα and Hβ of the ASN7 and ASP7 spin system and HN of SER8 spin systems. A table with chemical shift assignment of the protons for each spin system and xylose is also presented in Table 2.

FIGS. 4A-4B. NMR data. FIG. 4A) The TOCSY fingerprint region of OCF-S3 and OCF-S4. The spin system for ASP7 is no longer present, and a new spin system assigned as N-methylamide7 is present, indicating that OCF-I and OCF-J is fully converted during the EDC condensation reaction. Other amino acids present in occidiofungin are also labelled. Orange lines indicate unknown spin systems. FIG. 4B) NOESY data showing the NOEs indicating interaction of Hβ of N-methylamide7 to γ-NH and amide methyl group. Overlapping NOEs are seen indicating interaction of Hα of Gly6 to the HN of the N-methylamide7 spin system. NOEs not in this region are also which indicate interaction of Hα and Hβ of the N-methylamide7 spin system and HN of SER8. NOEs for other amino acids in occidiofungin are also labeled. A table with chemical shift assignment of the protons for each spin system and xylose is also presented in Table 3.

FIGS. 5A-5B. Reaction schematics for condensation of ASP7 (FIG. 5A) and GLU7 (FIG. 5B) occidiofungins with various amines. 10 molar equivalent of 1-ethyl-3-(3-dimethylaminopropyl) carbodiimide hydrochloride) (EDC), 1-hydroxybenzo-triazole (HOBt) and the amine is added to OCF-I, OCF-J, OCF-M, and OCF-N. The reaction was carried out in N,N-Dimethylformamide (DMF) or dimethyl sulfoxide (DMSO). The reaction was incubated at room temperature for at least 16 hours before separation using RP-HPLC.

FIGS. 6A-6F. Prodrug designs for occidiofungin. FIG. 6A) EDC condensation of OCF-I/OCF-J with an analog of 3-amino-1-propanol containing the prodrug moiety attached to the linker arm of the alcohol group (FIG. 6B). Occidiofungin analogs containing a prodrug moiety attached to the alcohol linker arm of OCF-S19/OCF-S20. The linker arm is metabolized by an enzyme to produce OCF-S19/OCF-S20. FIG. 6C) OCF-S21/OCF-S22 with a phosphate group attached to the linker arm. The phosphate group is removed upon incubation with alkaline phosphatase to produce OCF-S19/OCF-S20. FIG. 6D) EDC condensation of OCF-M/OCF-N with an analog of 3-amino-1-propanol containing the prodrug moiety attached to the linker arm of the alcohol group (FIG. 6E). Occidiofungin analogs containing a prodrug moiety attached to the alcohol linker arm of glutamic acid variants of OCF-S19/OCF-S20 synthesized from OCF-M and OCF-N. The linker arm is metabolized by an enzyme to produce glutamic acid variants of OCF-S19/OCF-S20. FIG. 6F) Glutamic acid variants of OCF-S21/OCF-S22 with a phosphate group attached to the linker arm. The phosphate group is removed upon incubation with alkaline phosphatase to produce glutamic acid variants of OCF-S19/OCF-S20.

FIG. 7. Overlay assay of S. cerevisiae DGY6. Spot A) 5 μg of OCF-S19/OCF-S20. Spot B) 5 μl of alkaline phosphatase reaction mixture (0.5 μl rCutsmart Buffer™, 0.25 μl Quick CIP, 4.25 μl ddH2O). Spot C) 5 μl of alkaline phosphatase reaction mixture post incubation for 90 minutes at 37° C. Spot D) 5 μg of OCF-S21/OCF-S22. Spot E) 5 μg of OCF-S21/OCF-S22 with alkaline phosphatase reaction mixture. Spot F) 5 μg of OCF-S21/OCF-S22 with alkaline phosphatase reaction mixture post incubation for 90 minutes at 37° C. Each spot was 5 μl volume for comparison purposes.

### BRIEF DESCRIPTION OF THE SEQUENCES

SEQ ID NO: 1: Sequence of cyclic occidiofungin antibiotic. Asn1-3 amino-5,6-dihydroxy-7-O-xylose-octadecanoic acid2-Ser3-BHY4-DABA5-Gly6-Asn7-Ser8].

SEQ ID NO: 2: Sequence of cyclic occidiofungin antibiotic. β-hydroxy Asn1-3 amino-5,6-dihydroxy-7-O-xylose-octadecanoic acid2-Ser3-BHY4-DABA5-Gly6-Asn7-Ser8].

SEQ ID NO: 3: Sequence of cyclic occidiofungin antibiotic. Asn1-3 amino-5,6-dihydroxy-7-O-xylose-octadecanoic acid2-Ser3-chloro-BHY4-DABA5-Gly6-Asn7-Ser8].

SEQ ID NO: 4: Sequence of cyclic occidiofungin antibiotic. β-hydroxy Asn1-3 amino-5,6-dihydroxy-7-O-xylose-octadecanoic acid2-Ser3-chloro-BHY4-DABA5-Gly6-Asn7-Ser8].

SEQ ID NO: 5: Sequence of cyclic occidiofungin antibiotic. Asn1-3 amino-5,6,7-trihydroxy-octadecanoic acid2-Ser3-BHY4-DABA5-Gly6-Asn7-Ser8].

SEQ ID NO: 6: Sequence of cyclic occidiofungin antibiotic. β-hydroxy Asn1-3 amino-5,6,7-trihydroxy-octadecanoic acid2-Ser3-BHY4-DABA5-Gly6-Asn7-Ser8].

SEQ ID NO: 7: Sequence of cyclic occidiofungin antibiotic. Asn1-3 amino-5,6,7-trihydroxy-octadecanoic acid2-Ser3-BHY4-DABA5-Gly6-Asp7-Ser8].

SEQ ID NO: 8: Sequence of cyclic occidiofungin antibiotic. β-hydroxy Asn1-3 amino-5,6,7-trihydroxy-octadecanoic acid2-Ser3-BHY4-DABA5-Gly6-Asp7-Ser8].

SEQ ID NO: 9: Sequence of cyclic occidiofungin antibiotic. Asn1-3 amino-5,6-dihydroxy-7-O-xylose-octadecanoic acid2-Ser3-BHY4-DABA5-Gly6-Asp7-Ser8].

SEQ ID NO: 10: Sequence of cyclic occidiofungin antibiotic. β-hydroxy Asn1-3 amino-5,6-dihydroxy-7-O-xylose-octadecanoic acid2-Ser3-BHY4-DABA5-Gly6-Asp7-Ser8].

SEQ ID NO: 11: Sequence of cyclic occidiofungin antibiotic. Asn1-3 amino-5,6-dihydroxy-7-O-xylose-octadecanoic acid2-Ser3-BHY4-DABA5-Gly6-Gln7-Ser8].

SEQ ID NO: 12: Sequence of cyclic occidiofungin antibiotic. β-hydroxy Asn1-3 amino-5,6-dihydroxy-7-O-xylose-octadecanoic acid2-Ser3-BHY4-DABA5-Gly6-Gln7-Ser8].

SEQ ID NO: 13: Sequence of cyclic occidiofungin antibiotic. Asn1-3 amino-5,6-30 dihydroxy-7-O-xylose-octadecanoic acid2-Ser3-BHY4-DABA5-Gly6-Glu7-Ser8].

SEQ ID NO: 14: Sequence of cyclic occidiofungin antibiotic. β-hydroxy Asn1-3 amino-5,6-dihydroxy-7-O-xylose-octadecanoic acid2-Ser3-BHY4-DABA5-Gly6-Glu7-Ser8].

### DETAILED DISCLOSURE OF THE INVENTION

As used herein, the singular forms “a”, “an” and “the” are intended to include the plural forms as well, unless the context clearly indicates otherwise. To the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrase “consisting essentially of” or “consists essentially of” indicates that the described embodiment encompasses embodiments containing the specified materials or steps and those that do not materially affect the basic and novel characteristic(s) of the described embodiment.

The term “about” means within an acceptable error range for the particular value as determined by one of ordinary skill in the art, which will depend in part on how the value is measured or determined, i.e., the limitations of the measurement system. In the context of the lengths of polynucleotides where the terms “about” are used, these polynucleotides contain the stated number of bases or base-pairs with a variation of 0-10% around the value (X±10%). In the context of compositions containing amounts of ingredients where the terms “about” or “approximately” are used, these compositions contain the stated amount of the ingredient with a variation (error range) of 0-10% around the stated value (X±10%).

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values.

When ranges are used herein, such as for dose ranges, combinations and subcombinations of ranges (e.g., subranges within the disclosed range), specific embodiments therein are intended to be explicitly included.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of an occidiofungin analog of the invention that is pharmaceutically acceptable and that possesses the desired pharmacological activity of the parent occidiofungin analog. In particular, such salts that are non-toxic may be inorganic or organic acid addition salts and base addition salts.

“Pharmaceutically acceptable vehicle” refers to a diluent, adjuvant, excipient or carrier with which an occidiofungin analog of the invention is administered. A “pharmaceutically acceptable vehicle” refers to a substance that is non-toxic, biologically tolerable, and otherwise biologically suitable for administration to a subject, such as an inert substance, added to a pharmacological composition or otherwise used to facilitate administration of an agent and that is compatible therewith. Examples of vehicles include but are not limited to calcium carbonate, calcium phosphate, various sugars and types of starch, cellulose derivatives, gelatin, vegetable oils, and polyethylene glycols.

“Subject” includes humans or non-human animals, particularly, mammals, such as bovine, porcine, canine, rodent, or feline animals.

“Treating” or “treatment” of any infection refers, in one embodiment, to ameliorating the infection or cancer (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the infection, either physically, (e.g., stabilization of a discernible symptom), physiologically, (e.g., stabilization of a physical parameter), or both. In yet another embodiment, “treating” or “treatment” refers to delaying the onset of the infection.

As used herein, the terms “reducing”, “inhibiting”, “blocking”, “preventing”, “alleviating”, or “relieving” when referring to an occidiofungin analog, mean that the occidiofungin analog brings down the occurrence, severity, size, volume or associated symptoms of an infection or cancer by at least about 7.5%, 10%, 12.5%, 15%, 17.5%, 20%, 22.5%, 25%, 27.5%, 30%, 35%, 40%, 45%, 50%, 55%, 60%, 65%, 70%, 75%, 80%, 90%, or 100% compared to how the infection or cancer would normally exist without application of the occidiofungin analog or a composition comprising the occidiofungin analog.

In certain embodiments, the methods according to the invention comprise the administration of a therapeutically effective amount of an occidiofungin analog to a subject suffering from or diagnosed as having an infection or cancer. A “therapeutically effective amount” means an amount or dose sufficient to generally bring about the desired therapeutic or prophylactic benefit in patients in need of such treatment for the designated infection or cancer.

Effective amounts or doses of the occidiofungin analogs of the present invention may be ascertained by routine methods such as modeling, dose escalation studies or clinical trials, and by taking into consideration routine factors, e.g., the mode or route of administration or drug delivery, the pharmacokinetics of the occidiofungin analog, the severity and course of the infection, the subject's previous or ongoing therapy, the subject's health status and response to drugs, and the judgment of the treating physician. An example of a dose is in the range of from about 0.001 to about 200 mg of an occidiofungin analog per kg of subject's body weight per day, preferably about 0.05 to 100 mg/kg/day, or about 1 to 35 mg/kg/day, even more preferably, about 1, 5, 10, or 20 mg/kg/day, in single or divided dosage units (e.g., BID, TID, QID). For a 70-kg human, an illustrative range for a suitable dosage amount is from about 0.05 to about 7 g/day, preferably, about 0.07 to about 2.45 g/day, even more preferably, about 0.07, 0.35, 0.7, or 1.4 g/day.

Structure of natural analogs of occidiofungin is provided by Formula (IX) and Formula (X):

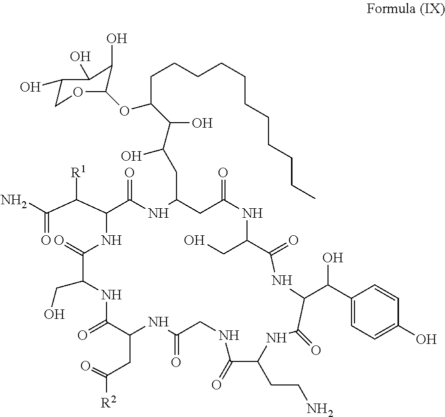

wherein R¹ is H or OH and R² is NH₂, OH.

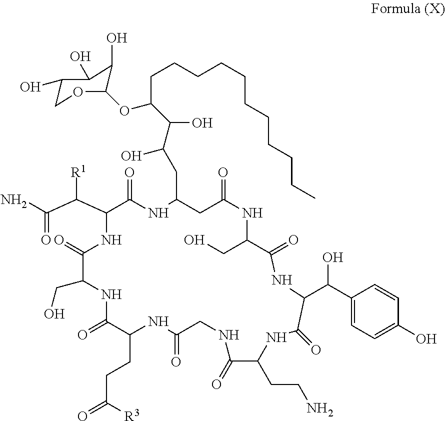

wherein R¹ is H or OH and R² is NH₂, OH.

As seen in Formula (IX) and Formula (X), natural analogs of occidiofungin are composed of eight amino acids (SEQ ID NOs: 1-14) and a C18 fatty amino acid (hereinafter, NAA) containing a xylose (xyl) sugar and a 2,4-diaminobutyric acid (DABA) (NAA is 3 amino-5,6-dihydroxy-7-O-xylose-octadecanoic acid and is found in position 2 of SEQ ID NOs: 1-4 and 9-14). The structural analogs of Formula (IX) and Formula (X) differ by the addition of oxygen to asparagine 1 (Asn1) forming a β-hydroxy asparagine 1 (BHN1), and the amino acid residue at position 7. For Formula (IX), occidiofungin analogs OCF-A (SEQ ID NO: 1) and OCF-B (SEQ ID NO: 2) have an asparagine residue at position 7. OCF-A (1200.59 Da) has an asparagine residue at position 1 and OCF-B (1216.59 Da) has a β-hydroxy asparagine at position 1. OCF-I (SEQ ID NO: 9) and OCF-J (SEQ ID NO: 10) has an aspartic acid residue at position 7, and a hydrogen or hydroxy group at position 1, respectively. For Formula (X), OCF-K (SEQ ID NO: 11) and OCF-L (SEQ ID NO: 12) have a glutamine residue at position 7. OCF-K has an asparagine residue at position 1 and OCF-L (1230.61 Da) has a β-hydroxy asparagine at position 1. OCF-M (SEQ ID NO: 3) and OCF-N(SEQ ID NO: 14) has a glutamic acid residue at position 7. OCF-M has an asparagine residue at position 1 and OCF-N (1231.59 Da) has a β-hydroxy asparagine at position 1 respectively. HRMS analysis was performed to confirm the identity of the occidiofungin analogs (Table 4). The occidiofungin aspartic acid 7 variants (OCF-I and OCF-J) and glutamic acid variants (OCF-M and OCF-N) were isolated using reversed-phase high-performance liquid chromatography (RP-HPLC) allowing for their resolution with the previously characterized asparagine 7 variants (OCF-A and OCF-B) (Table 1). OCF-K and OCF-L co-elutes with OCF-A and OCF-B, and OCF-M and OCF-N co-elutes with OCF-I and OCF-J by RP-HPLC. Nuclear magnetic resonance (NMR) analyses confirmed the variant to contain aspartic acid or glutamic acid on residue 7.

Certain embodiments of the instant invention provide occidiofungin analogs and methods of making occidiofungin analogs.

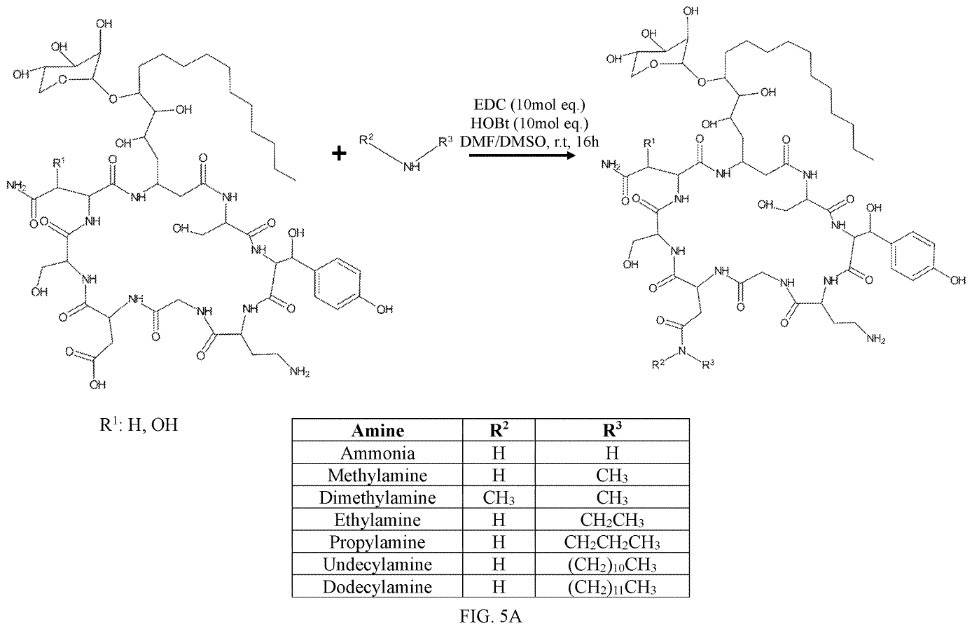

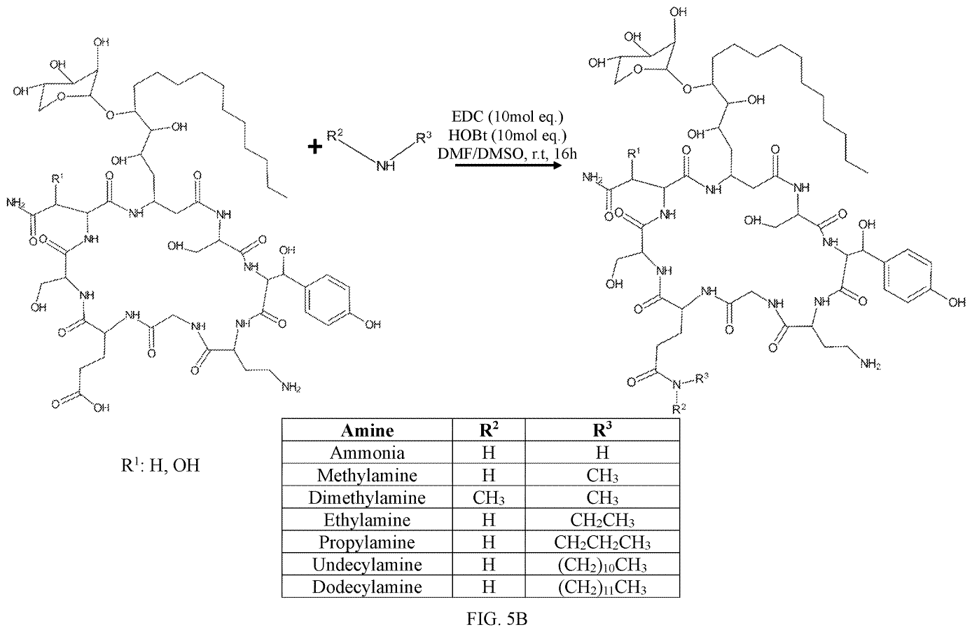

OCF-I, OCF-J, OCF-M and OCF-N variants present a novel site for analog production as the terminal side chain in ASP7 or GLU7 is the sole carboxylic acid in the compound. The single carboxylic acid in the molecule provides a selective region for modifying the natural product using a variety of synthetic chemistry approaches. In certain embodiments, selective modification has been performed by using a carbodiimide, such as, for example, EDC (1-ethyl-3-(3-dimethylaminopropyl) carbodiimide hydrochloride, N,N′-Dicyclohexyl carbodiimide (DCC), or N,N′-Diisopropylcarbodiimide (DIC), mediated condensation with a variety of amines to form an amide bond between the carboxylic acid at position 7 (FIG. 5A, Table 5). All semi-synthetic analogs were confirmed by mass spectrometry and NMR analysis.

In certain embodiments, the carbodiimide mediated condensation reaction is a simple, selective, one-step reaction that can be used for high throughput semi-synthetic analog production. This carbodiimide coupling reaction has been performed with a variety of primary and secondary amines on the carboxylic acid of ASP7 or GLU7 side chain. In certain embodiments, the amine is ammonia, methylamine, dimethylamine, ethylamine, propylamine, butylamine, pentylamine, hexylamine, heptylamine, octylamine, nonylamine, decylamine, undecylamine, or dodecylamine, but-3-yn-1-amine, 3-amino-1-propanol, 3-aminopropyl-dihydrogen-phosphate, an amine (e.g., 3-amino-1-propanol) conjugated to a prodrug function group, such as, for example, a phosphate group or methyl or benzyl succinate, or any primary or secondary amine with various alkyl and/or aryl group(s). In certain embodiments, 1-hydroxybenzo-triazole (HOBt), 4-(dimethylamino)pyridine (DMAP), N-hydroxysulfosuccinimide (Sulfo-NHS), Bis(dimethylamino)methylene]-1H-1,2,3-triazolo[4,5-b]pyridinium 3-oxide (HATU), or any combination thereof is added to the condensation reaction. In certain embodiments, the condensation reaction is carried out in N,N-Dimethylformamide (DMF), dimethyl sulfoxide (DMSO), tetrahydrofuran (THF), methyl tert-butyl ether (TBME), cyclopentyl methyl ether (CPME), dichloromethane, dimethyl carbonate (DMC), ethyl acetate (EtOAc), or isopropyl alcohol (IPA). In certain embodiments, a 10× excess molar ratio of the desired amine, EDC, and 1-Hydroxybenzotriazole (HOBt) are used, relative to the occidiofungin analog, in, for example, DMF or DMSO at room temperature (e.g., about 18° C. to about 25° C.) for about 16, 18, 20, 22, 24, 26, 28, 30, 36, or 48 hours or longer, and, preferably, in the absence of light. In certain embodiments, a longer incubation time and/or incubating at about 35° C. has shown increased efficiency for some of the amines used in the reaction, particularly the larger amines.

In certain embodiments, a crosslinking catalyst, such as, for example, N-Hydroxysuccinimide, can activate the carboxyl group, and the activated occidiofungin intermediate can be used to make additional analogs in a high-throughput combinatorial chemistry to rapidly generate large libraries of analogs.

In certain embodiments, additional chemistry, multicomponent reactions such as Ugi and Passerini reactions, are available to take advantage of the ASP7 (OCF-I and OCF-J) or GLU7 (OCF-M and OCF-N) occidiofungin variant containing the carboxylic acid modification site. In certain embodiments, the Ugi reaction can comprise, for example, OCF-I or OCF-J reacted with a primary or secondary amine (e.g., such as the amines disclosed above), methyl isocyanide, and an aldehyde to yield the corresponding Ugi condensation product.
OCF-I and OCF-J reactants and products follow the scheme:

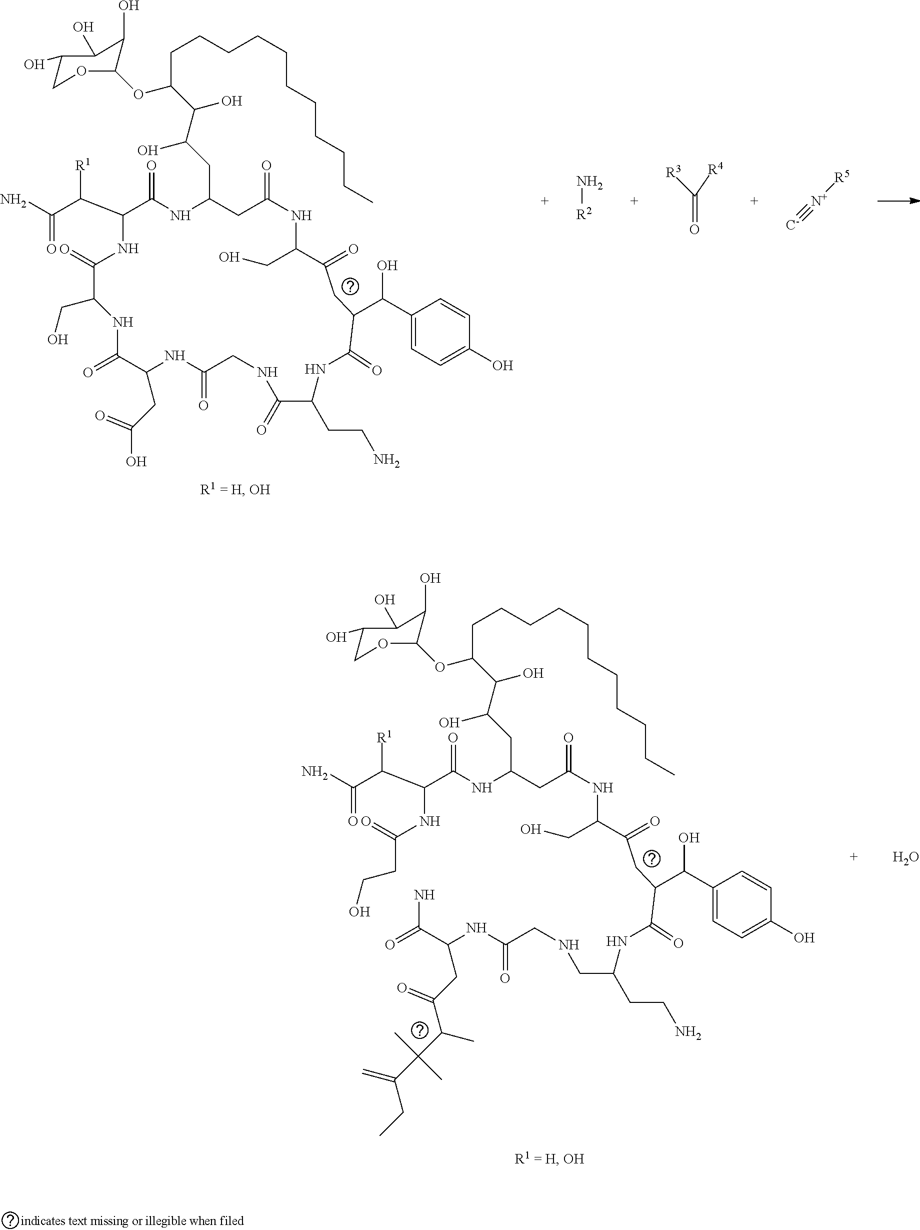

OCF-M and OCF-N reactants and products follow the scheme:

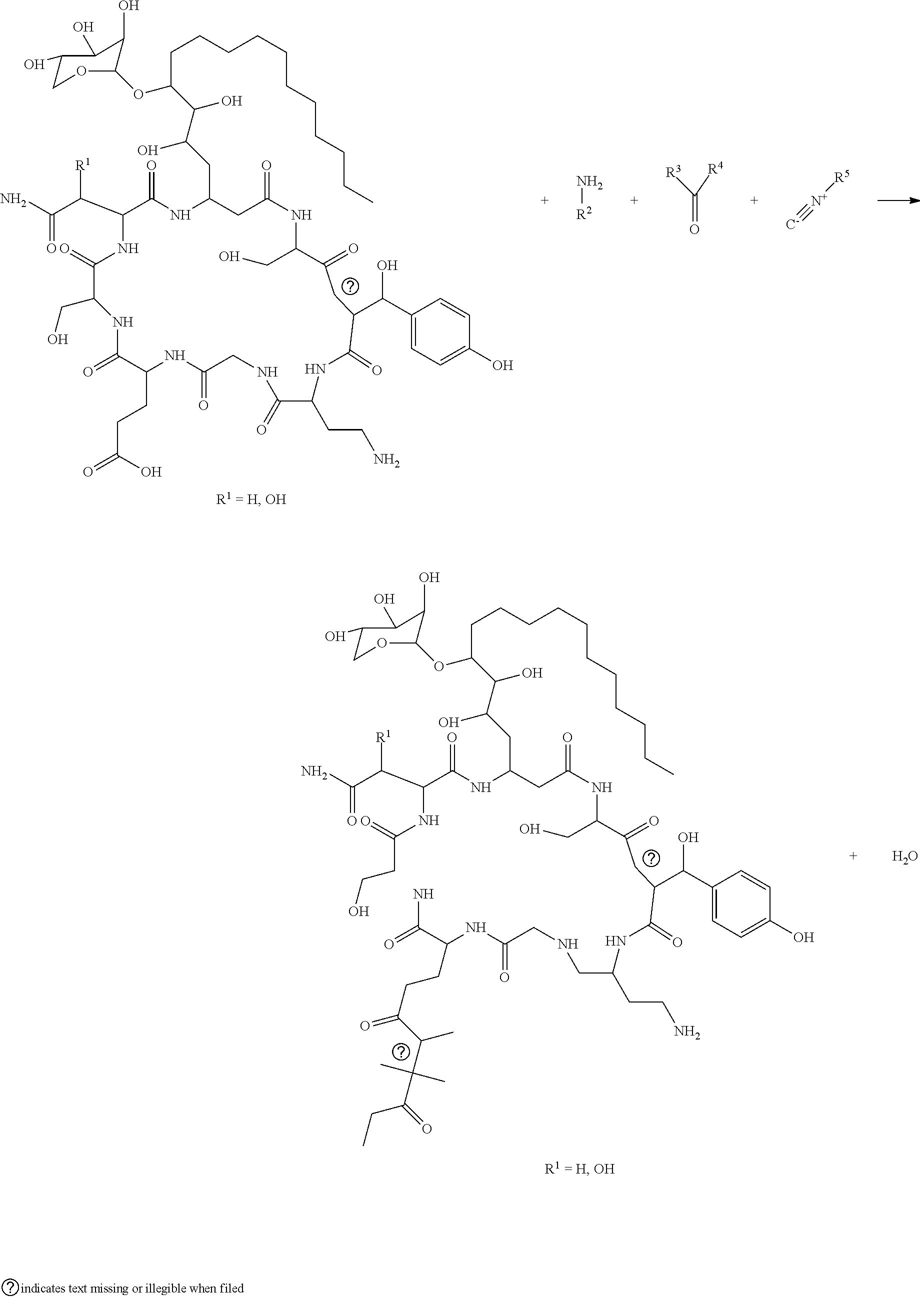

In certain embodiments, the Passerini reaction can comprise, for example, OCF-I or OCF-J reacted with methyl isocyanide and an aldehyde or ketone to yield the corresponding Passerini condensation product.
OCF-I and OCF-J reactants and products follow the scheme:

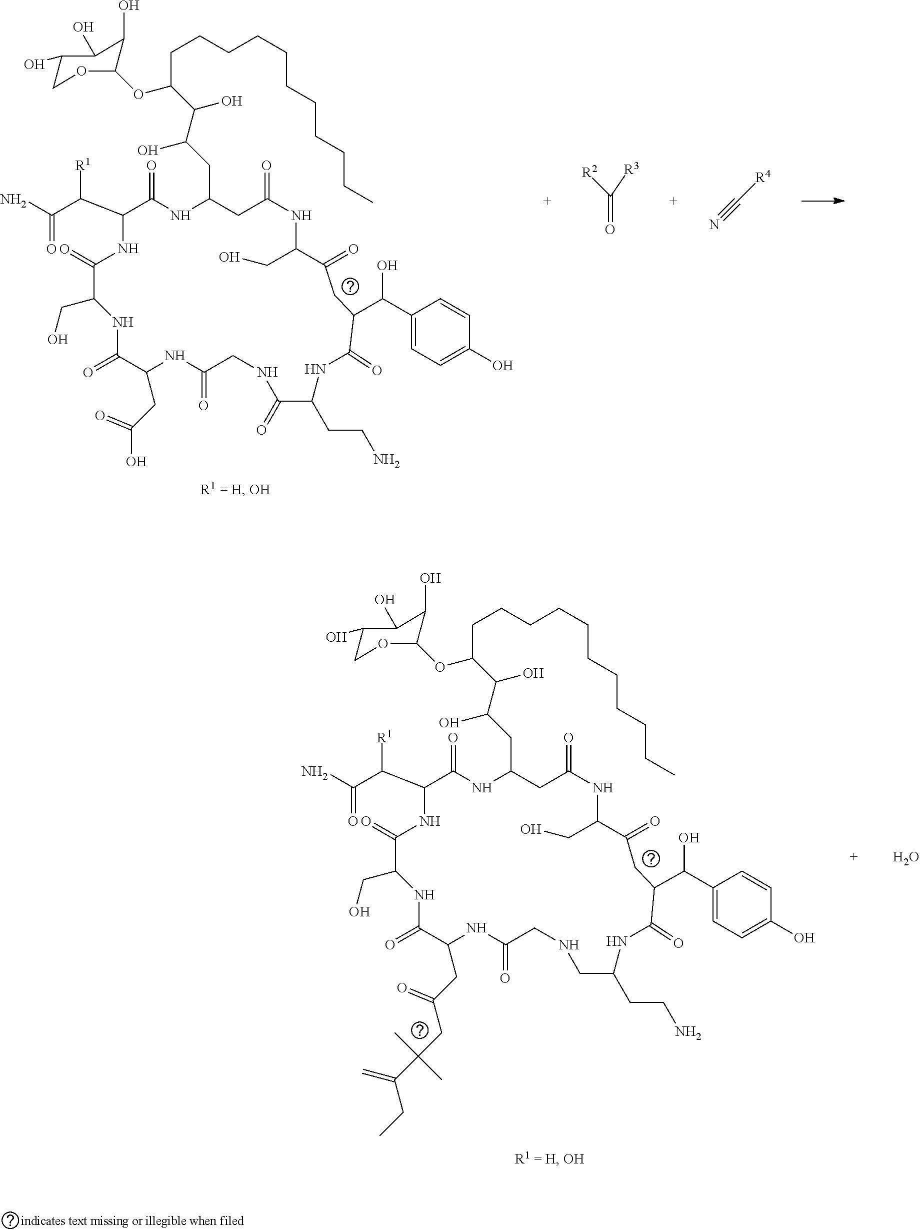

OCF-M and OCF-N reactants and products follow the scheme:

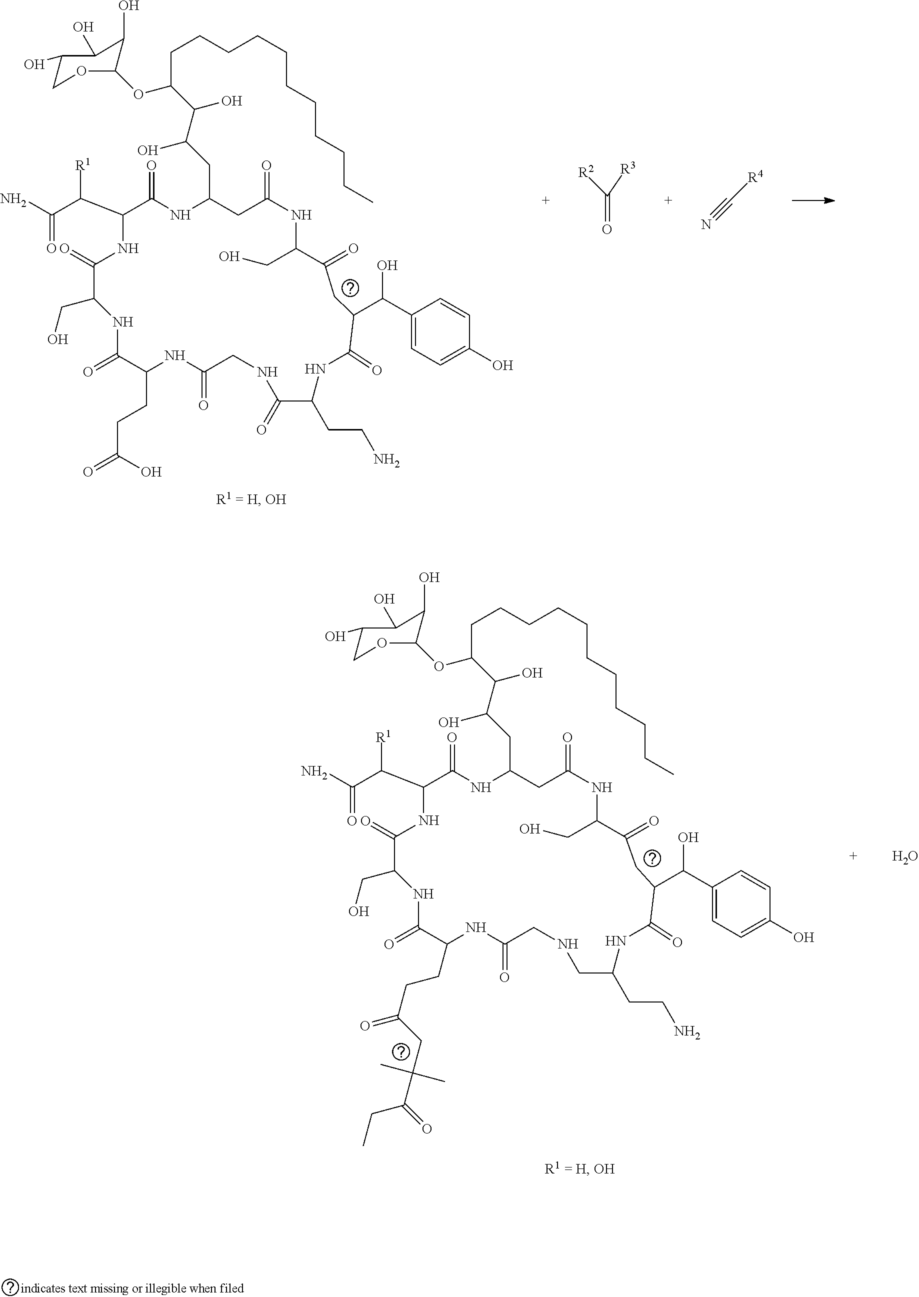

In certain embodiments, an occidiofungin analog is produced using carbodiimide condensation with an amine, and it can be represented by Formula (I), Formula (II), Formula (III), or Formula (IV):

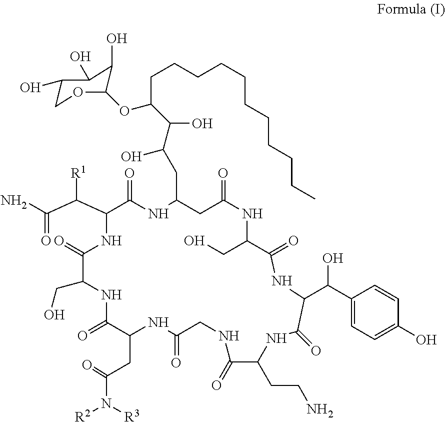

wherein R¹ is H, R² is H or CH₃, and R³ is H, CH₃, CH₂CH₃, CH₂CH₂CH₃, (CH₂)₁₀CH₃, or (CH₂)₁₁CH₃. In preferred embodiments, R¹ is H or OH, R² is H, and R³ is H, CH₃, CH₂CH₃, CH₂CH₂CH₃, (CH₂)₁₀CH₃, or (CH₂)₁₁CH₃; or R¹ is H or OH, R² is CH₃, and R³ is CH₃

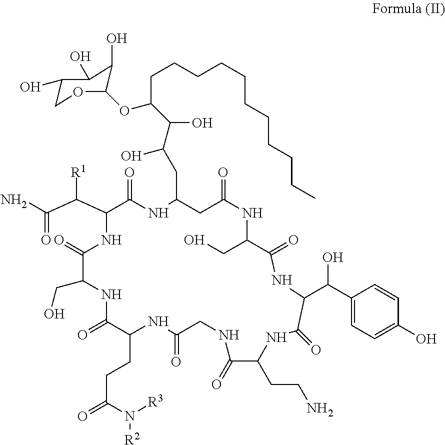

wherein R¹ is H or OH, R² is H or CH₃, and R³ is H, CH₃, CH₂CH₃, CH₂CH₂CH₃, (CH₂)₁₀CH₃, or (CH₂)₁₁CH₃. In preferred embodiments, R¹ is H or OH, R² is H, and R³ is H, CH₃, CH₂CH₃, CH₂CH₂CH₃, (CH₂)₁₀CH₃, or (CH₂)₁₁CH₃; or R¹ is H or OH, R² is CH₃, and R³ is CH₃

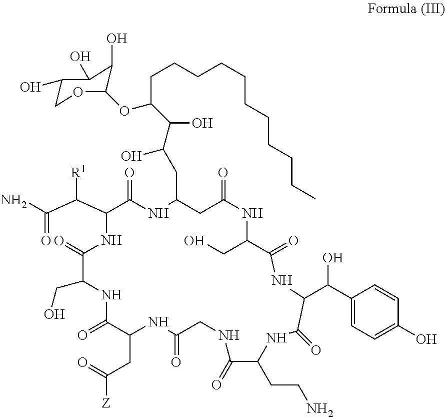

wherein R¹ is H or OH, and Z is an ester, a thioester, an acid anhydride, an acid chloride, an amide or a carboxylate ion

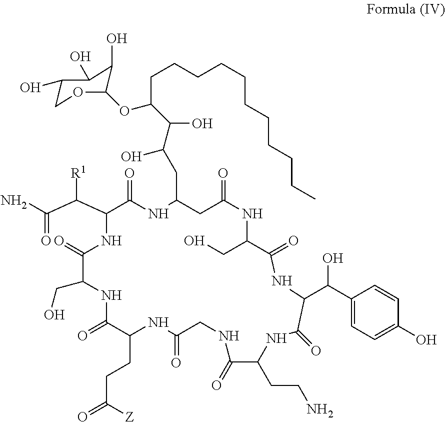

wherein R¹ is H or OH, and Z is an ester, a thioester, an acid anhydride, an acid chloride, an amide or a carboxylate ion.

In certain embodiments, carbodiimide (e.g., EDC) mediated condensation of OCF-I and OCF-J with ammonia affords OCF-A and OCF-B variants (named OCF-S1 and OCF-S2), by converting ASP7 to ASN7 (FIG. 5A, Table 5). Observation of electrospray ionization mass spectrometry (ESI-MS) data from the ammonia reaction restored OCF-A and OCF-B variants, but an additional product was also being made with a mass of 1183.56 Da and 1998.57 Da (OCF-S15 and OCF-S16). The mass of these variants in the reaction correspond to an unwanted dehydration side-product of OCF-I and OCF-J, named OCF-S15 and OCF-S16. The side-product is not observed by mass spectrometry in OCF-M and OCF-N (GLU7 variants) due to their low abundance.

In certain embodiments, additional amines can be used in a condensation reaction, preferably amines that are linked to prodrug function groups. In preferred embodiments, the prodrug functional group is a methyl succinate, benzyl succinate, ether, a phosphate group, or any other recognized prodrug function groups, including a prodrug function group disclosed in Rautio J, Kumpulainen H, Heimbach T, Oliyai R, Oh D, Järvinen T, Savolainen J. Prodrugs: design and clinical applications. Nat Rev Drug Discov. 2008 March; 7 (3): 255-70. doi: 10.1038/nrd2468. Erratum in: Nat Rev Drug Discov. 2008 March; 7 (3): 272, which is hereby incorporated by reference in its entirely.

In certain embodiments, direct condensation may not be achieved using an amine, such as, for example, ethanolamine, 3-amino-1-butanol, 4-amino-1-butanol, 3-amino-1-propanol, 3-aminopropyl-dihydrogen-phosphate, or higher alkyl chain amino alcohols (e.g., n-amino-n alkyl-1-ol). Therefore, a portion of the amine, such as, for example, an alcohol group of amine can be protected before condensation. In certain embodiments, 3-amino-1-propanol, is protected with tert-Butyldimethylsilyl (TBS) by adding, for example, 1 molar equivalence of 3-amino-1-propanol, triethylamine and tert-Butyldimethylsilyl chloride (TBS-C1) in dichloromethane (DCM). In certain embodiments, the reaction mixture is mixed and incubated at room temperature for at least about 4, 5, 6, 7, 8, 9, 10, or more hours. The oily layer produced after incubation contains the protected 3-amino-1-propanol, and is used as the amine in the condensation reaction. In certain embodiments, 1 molar equivalence of 3-aminopropyl-dihydrogen-phosphate, triethylamine and TBS-Cl is incubated in DCM. The reaction mixture is mixed and equal volume of 35% acetonitrile (65% double distilled water) 0.1% trifluoroacetic acid is added, and is then incubated at room temperature for at least about 4, 5, 6, 7, 8, 9, 10, or more hours. The top aqueous layer contains the protected 3-aminopropyl-dihydrogen-phosphate and is subsequently used in the condensation reaction.

In certain embodiments, prodrug functional groups, such as, for example, methyl succinate or benzyl succinate can be conjugated to an amine before the condensation reaction. In preferred embodiments, tert-butoxycarbonyl (Boc) 3-amino-1-propanol (Boc-3-amino-1-propanol), allyloxy carbonyl (Alloc) 3-amino-1-propanol (Boc-3-amino-1-propanol), or benzyloxycarbonlyl (Boc) 3-amino-1-propanol (Boc-3-amino-1-propanol) was first conjugated to the methyl or benzyl succinate. In certain embodiments, alternatives to the Boc hydroxy protection group, include, for example, methoxymethyl ether, tetrahydropyranyl ether, allyl ether, benzyl ether, t-butyldimethylsilyl ether, t-butyldiphenylsilyl ether, acetic acid ester, pivalic acid ester, or benzoic acid ester. In preferred embodiments, 1 molar equivalence of 3-amino-1-propanol, N,N′-Dicyclohexylcarbodiimide (DCC), methyl succinate/benzyl succinate and 0.1 molar equivalence of 4-dimethylaminopyridine (DMAP) was added to anhydrous dimethylformamide (DMF). In certain embodiments, the reaction was stirred at room temperature for at least about 12, 13, 14, 15, 16, 17, 18, 19, 20, or more hours. In certain embodiments, the prodrug functional group conjugated to an amine can be further purified from the reaction mixture.

In certain embodiments, an amine, such as, for example, 3-amino-1-propanol, is used to make an occidiofungin analog (according to Formula (V) and Formula (VI)), such, as, for example, OCF-S19 and OCF-S20, containing a terminal alcohol group (Table 5). In certain embodiments, the terminal alcohol group of the occidiofungin analog links the occidiofungin to a prodrug functional group, such as, for example, a phosphate group, methyl succinate, an ether or benzoic acid ester.

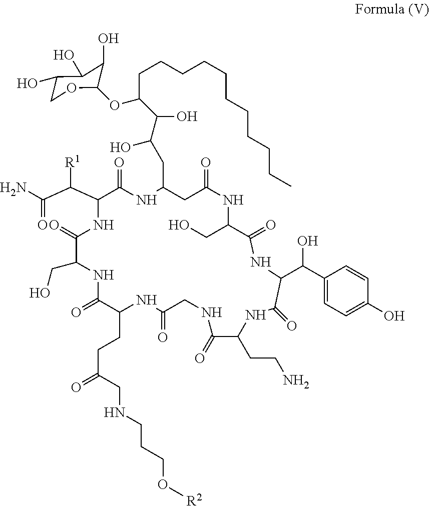

wherein R¹ is H or OH and R² is a prodrug-functional group;

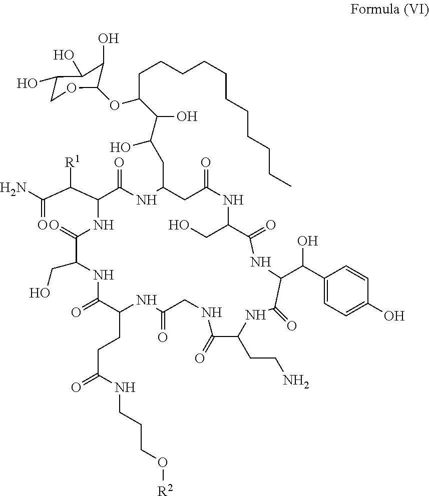

wherein R¹ is H or OH and R² is a prodrug-functional group.

In certain embodiments, the prodrug functional group acts as a prodrug moiety in which chemical or enzymatic action can remove the prodrug functional group. In certain embodiments, the prodrug is activated by enzymes or chemicals in the subject, such as, for example, enzymes or chemicals found in blood or enzymes or chemicals found in cells. In certain embodiments, the enzyme is a phosphatase, esterase, or protease. In certain embodiments, the prodrug functional group acts as a prodrug moiety in which chemical or enzymatic removal of the prodrug functional group results in OCF-S19 and OCF-S20. Additional prodrug moieties can use a linker, leaving a group at the ASP7 position using a phosphatase, esterase, or protease.

In certain embodiments, an amine, such as, for example, but-3-yn-1-amine, can be used to make an occidiofungin analog (according to Formula (I) and Formula (II)), such, as, for example, OCF-S23 and OCF-S24, containing a terminal alkyne group (Table 5). In certain embodiments, the terminal alkyne group of the occidiofungin analog can be used in subsequent Click reaction chemistry to link the occidiofungin to a functional group, such as, for example, an azide functional group.

In certain embodiments, the analog of occidiofungin is according to Formula (VII) and Formula (VIII):

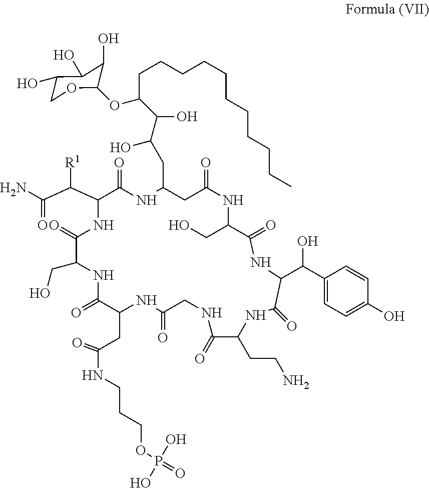

wherein R¹ is H or OH;

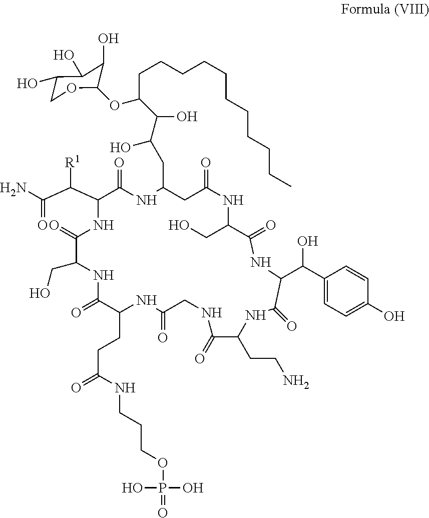

wherein R¹ is H or OH.

In certain embodiments, additional amines can be used in a condensation reaction, preferably amines that function as antibodies or are linked to antibodies. In preferred embodiments, the antibody is a monoclonal antibody. In certain embodiments, the ASP7 residue is modified using the aforementioned condensation reaction to contain a linker conjugated to a monoclonal antibody enabling a targeting-based delivery system for specific cancer cells.

In certain embodiments, the antibody can target at least one antigen, including, for example, CD19, CD3E, ERBB2 (HER2), EGFR, MS4A1 (CD20), CD22, PDCD1 (PD-1), MSLN (mesothelin), and ERBB3 (Her3), Th17 cytokine, IL17A, or any other antigen that is currently targeted by an antibody for the treatment of cancer or any other disease, as disclosed by Strohl W R. Current progress in innovative engineered antibodies. Protein Cell. 2018 January; 9 (1): 86-120. doi: 10.1007/s13238-017-0457-8. Epub 2017 Aug. 18, which is hereby incorporated by reference in its entirety. In certain embodiments, the antibody is muromonab-CD3, abciximab, rituximab, daclizumab, infliximab, palivizumab, trastuzumab, etanercept, basiliximab, gemtuzumab ozogamicin, alemtuzumab, ibritumomab tiuxetan, adalimumab, alefacept, omalizumab, tositumomab-i131, efalizumab, cetuximab, bevacizumab, natalizumab, abatacept, ranibizumab, panitumumab, eculizumab, rilonacept, romiplostim, golimumab, ustikinumab, catumaxomab, certolizumab pegol, canakinumab, ofatumumab, tocilizumab, denosumab, belimumab, ipilimumab, belatacept, brentuximab vedotin, aflibercept, mogamulizumab, pertuzumab, ziv-aflibercept, raxibacumab, trastuzumab emtansine, obinutuzumab, eftrenonacog alfa, ramucirumab, siltuximab, vedolizumab, efmoroctocog alfa, pembrolizumab, dulaglutide, alemtuzumab, blinatumomab, nivolumab, secukinumab, dinutuximab, alirocumab, evolocumab, idarucizumab, asfotase alfa, mepolizumab, daratumumab, necitumumab, elotuzumab, obiltoxaximab, ixekizumab, reslizumab, atezolizumab, daclizumab, olaratumab, bezlotoxumab, brodalumab, avelumab, dupilumab, ocrelizumab, durvalumab, any other antibody disclosed by Strohl WR, 2017, or any combination thereof.

### Salts of Occidiofungin Analogs of the Invention

In some embodiments the subject invention provides salts of the occidiofungin analogs described herein. The salts can be a salt with an inorganic acid, such as hydrochloric acid, hydrobromic acid, perchloric acid, nitric acid, thiocyanic acid, sulfuric acid, and phosphoric acid; an organic acid, such as trifluoroacetic acid (TFA), formic acid, acetic acid, propionic acid, glycolic acid, lactic acid, pyruvic acid, oxalic acid, malonic acid, succinic acid, maleic acid, and fumaric acid; or a salt with a base, such as sodium hydroxide, ammonium hydroxide, potassium hydroxide, and organic bases such as mono-, di-, trialkyl and aryl amines, and substituted ethanolamines.

Further salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-di sulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent occidiofungin analogue is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the occidiofungin analogs contains a basic functionality, salts of non-toxic organic or inorganic acids, such as hydrochloride, hydrobromide, tartrate, mesylate, acetate, maleate, oxalate and the like.

Certain embodiments provide amorphous forms of salts of the occidiofungin analogs disclosed herein. Such amorphous forms are advantageous for oral, pulmonary, buccal, intravaginal, or suppository delivery.

### Routes of Administration and Dosage Forms

In certain embodiments, the occidiofungin analogs can be administered intramuscularly, subcutaneously, intrathecally, intravenously or intraperitoneally by infusion or injection. Solutions of the occidiofungin analogs can be prepared in water, optionally mixed with a nontoxic surfactant. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the occidiofungin analogs that are adapted for the extemporaneous preparation of sterile injectable or infusible solutions or dispersions, optionally encapsulated in liposomes. Preferably, the ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, cyclodextrins, and suitable mixtures thereof. The proper fluidity can be maintained by, for example, the formation of liposomes, by the maintenance of the required particle size in the case of dispersions, or by the use of surfactants. The prevention of the action of microorganisms can be brought about by various antibacterial and antifungal agents, for example, parabens, chlorobutanol, phenol, sorbic acid, thimerosal, and the like. In many cases, it is preferable to include isotonic agents, for example, sugars, buffers, or sodium chloride. Prolonged absorption of the injectable compositions can be brought about by the use in the compositions of agents delaying absorption, for example, aluminum monostearate and gelatin.

Sterile injectable solutions are prepared by incorporating the occidiofungin analogs in the required amount in the appropriate solvent as described herein with various of the other ingredients enumerated herein, as required, preferably followed by filter sterilization. In the case of sterile powders for the preparation of sterile injectable solutions, the preferred methods of preparation are vacuum drying and freeze drying techniques, which yield a powder of the active ingredient plus any additional desired ingredient present in the previously sterile-filtered solutions.

The compositions of the subject invention may also be administered orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the subject's diet.

For oral therapeutic administration, the occidiofungin analogs can be combined with one or more excipients and used in the form of ingestible tablets, buccal tablets, troches, capsules, elixirs, suspensions, syrups, wafers, and the like. Such compositions and preparations should contain at least 0.1% of an occidiofungin analog of the present invention. The percentage of the occidiofungin analogs of the invention present in such compositions and preparations may, of course, be varied and may conveniently be between about 2% to about 60% of the weight of a given unit dosage form. The amount of the occidiofungin analogs in such therapeutically useful compositions is such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid, and the like; a lubricant such as magnesium stearate; and a sweetening agent such as sucrose, fructose, lactose, or aspartame, or a flavoring agent such as peppermint, oil of wintergreen, or cherry flavoring may be added.

When the unit dosage form is a capsule, it may contain, in addition to materials of the above type, a liquid carrier, such as a vegetable oil or a polyethylene glycol.

Various other materials may be present as coatings or for otherwise modifying the physical form of the solid unit dosage form. For instance, tablets, pills, or capsules may be coated with gelatin, wax, shellac, or sugar, and the like. A syrup or elixir may contain the occidiofungin analog, sucrose or fructose as a sweetening agent, methyl and propylparabens as preservatives, a dye, and flavoring such as cherry or orange flavor.

Of course, any material used in preparing any unit dosage form should be pharmaceutically acceptable and substantially non-toxic in the amounts employed.

In addition, the occidiofungin analogs may be incorporated into sustained-release preparations and devices. For example, the occidiofungin analogs may be incorporated into time release capsules, time release tablets, time release pills, and time release occidiofungin analogs or nanoparticles.

Pharmaceutical compositions for topical administration of the occidiofungin analogs to the epidermis (mucosal or cutaneous surfaces) can be formulated as ointments, creams, lotions, gels, or as a transdermal patch. Such transdermal patches can contain penetration enhancers such as linalool, cyclodextrins, carvacrol, thymol, citral, menthol, t-anethole, and the like. Ointments and creams can, for example, include an aqueous or oily base with the addition of suitable thickening agents, gelling agents, colorants, and the like. Lotions and creams can include an aqueous or oily base and typically also contain one or more emulsifying agents, stabilizing agents, dispersing agents, suspending agents, thickening agents, coloring agents, and the like. Gels preferably include an aqueous carrier base and include a gelling agent such as cross-linked polyacrylic acid polymer, a derivatized polysaccharide (e.g., carboxymethyl cellulose), and the like.

Pharmaceutical compositions suitable for topical administration in the mouth (e.g., buccal or sublingual administration) include lozenges comprising the composition in a flavored base, such as sucrose, acacia, or tragacanth; pastilles comprising the composition in an inert base such as gelatin and glycerin or sucrose and acacia; and mouthwashes comprising the active ingredient in a suitable liquid carrier. The pharmaceutical compositions for topical administration in the mouth can include penetration enhancing agents, if desired.

Useful solid carriers include finely divided solids such as talc, clay, microcrystalline cellulose, silica, alumina, and the like. Other solid carriers include nontoxic polymeric nanoparticles or microparticles. Useful liquid carriers include water, alcohols, or glycols, or water/alcohol/glycol blends, in which the occidiofungin analogs can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of useful dermatological compositions which can be used to deliver the occidiofungin analogs to the skin are known in the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508), all of which are hereby incorporated by reference.

The concentration of the occidiofungin analogs of the invention in such formulations can vary widely depending on the nature of formulation and intended route of administration. For example, the concentration of the occidiofungin analogs in a liquid composition, such as a lotion, can preferably be from about 0.1-25% by weight, or, more preferably, from about 0.5-10% by weight. The concentration in a semi-solid or solid composition such as a gel or a powder can preferably be about 0.1-5% by weight, or, more preferably, about 0.5-2.5% by weight.

Pharmaceutical compositions for spinal administration or injection into amniotic fluid can be provided in unit dose form in ampoules, pre-filled syringes, small volume infusion, or in multi-dose containers, and can include an added preservative. The compositions for parenteral administration can be suspensions, solutions, or emulsions, and can contain excipients such as suspending agents, stabilizing agents, and dispersing agents.

A pharmaceutical composition suitable for rectal administration comprises occidiofungin analogs of the present invention in combination with a solid or semisolid (e.g., cream or paste) carrier or vehicle. For example, such rectal compositions can be provided as unit dose suppositories. Suitable carriers or vehicles include cocoa butter and other materials commonly used in the art.

According to one embodiment, pharmaceutical compositions of the present invention suitable for vaginal administration are provided as pessaries, tampons, creams, gels, pastes, foams, or sprays containing an occidiofungin analog of the invention in combination with carriers as are known in the art, including, for example, cyclodextrin. Alternatively, compositions suitable for vaginal administration can be delivered in a liquid or solid dosage form.

Pharmaceutical compositions suitable for intra-nasal administration are also encompassed by the present invention. Such intra-nasal compositions comprise an occidiofungin analog of the invention in a vehicle and suitable administration device to deliver a liquid spray, dispersible powder, or drops. Drops may be formulated with an aqueous or non-aqueous base also comprising one or more dispersing agents, solubilizing agents, or suspending agents. Liquid sprays are conveniently delivered from a pressurized pack, an insufflator, a nebulizer, or other convenient means of delivering an aerosol comprising an occidiofungin analog. Pressurized packs comprise a suitable propellant such as dichlorodifluoromethane, trichlorofluoromethane, dichlorotetrafluoroethane, carbon dioxide, or other suitable gas as is well known in the art. Aerosol dosages can be controlled by providing a valve to deliver a metered amount of an occidiofungin analog.

The occidiofungin analogs may be combined with an inert powdered carrier and inhaled by the subject or insufflated.

Pharmaceutical compositions for administration by inhalation or insufflation can be provided in the form of a dry powder composition, for example, a powder mix of an occidiofungin analog and a suitable powder base such as lactose or starch. Such powder composition can be provided in unit dosage form, for example, in capsules, cartridges, gelatin packs, or blister packs, from which the powder can be administered with the aid of an inhalator or insufflator.

The exact amount (effective dose) of the occidiofungin analogs varies from subject to subject, depending on, for example, the species, age, weight, and general or clinical condition of the subject, the severity or mechanism of any infection being treated, the particular agent or vehicle used, the method and scheduling of administration, and the like. A therapeutically effective dose can be determined empirically, by conventional procedures known to those of skill in the art. See, e.g., The Pharmacological Basis of Therapeutics, Goodman and Gilman, eds., Macmillan Publishing Co., New York. For example, an effective dose can be estimated initially either via in vivo assays or in suitable animal models. The animal model may also be used to determine the appropriate concentration ranges and routes of administration. Such information can then be used to determine useful doses and routes for administration in humans. Methods for the extrapolation of effective dosages in mice and other animals to humans are known to the art; for example, see U.S. Pat. No. 4,938,949, which is hereby incorporated by reference. A therapeutic dose can also be selected by analogy to dosages for comparable therapeutic agents.

The particular mode of administration and the dosage regimen can be selected by the attending clinician, taking into account the particulars of the case (e.g., the subject, the disease, the disease state involved, and whether the treatment is prophylactic). Treatment may involve daily or multi-daily doses of compound(s) over a period of a few days to months, or even years.

In general, however, a suitable dose can be in the range of from about 0.001 to about 100 mg/kg of body weight per day, preferably from about 0.01 to about 100 mg/kg of body weight per day, more preferably, from about 0.1 to about 50 mg/kg of body weight per day, or even more preferred, in a range of from about 1 to about 10 mg/kg of body weight per day. For example, a suitable dose may be about 1 mg/kg, 10 mg/kg, or 50 mg/kg of body weight per day.

The occidiofungin analogs can be conveniently administered in unit dosage form, containing for example, about 0.05 to about 10000 mg, about 0.5 to about 10000 mg, about 5 to about 1000 mg, or about 50 to about 500 mg of active ingredient per unit dosage form.

The occidiofungin analogs can be administered to achieve peak plasma concentrations of, for example, from about 0.25 to about 200 μM, about 0.5 to about 75 μM, about 1 to about 50 μM, about 2 to about 30 μM, or about 5 to about 25 μM. Exemplary desirable plasma concentrations include at least 0.25, 0.5, 1, 5, 10, 25, 50, 75, 100 or 200 μM. For example, plasma levels may be from about 1 to about 100 micromolar or from about 10 to about 25 micromolar. This may be achieved, for example, by the intravenous injection of a 0.05 to 5% solution of an occidiofungin analog, optionally in saline, or orally administered as a bolus containing about 1 to about 100 mg of the occidiofungin analog. Desirable blood levels may be maintained by continuous or intermittent infusion.

The occidiofungin analogs can be included in the compositions within a therapeutically useful and effective concentration range, as determined by routine methods that are well known in the medical and pharmaceutical arts. For example, a typical composition can include one or more of the occidiofungin analogs at a concentration in the range of at least about 1 mg/ml, preferably at least about 4 mg/ml, more preferably at least 5 mg/ml and most preferably at least 6 mg/ml.

The occidiofungin analogs may conveniently be presented in a single dose or as divided doses administered at appropriate intervals, for example, as one dose per day or as two, three, four or more sub-doses per day. The sub-dose itself may be further divided, e.g., into a number of discrete loosely spaced administrations, such as multiple inhalations from an insufflator.

Optionally, the pharmaceutical compositions of the present invention can include one or more other therapeutic agents, e.g., as a combination therapy. The additional therapeutic agent(s) will be included in the compositions within a therapeutically useful and effective concentration range, as determined by routine methods that are well known in the medical and pharmaceutical arts. The concentration of any particular additional therapeutic agent may be in the same range as is typical for use of that agent as a monotherapy, or the concentration may be lower than a typical monotherapy concentration if there is a synergy when combined with an occidiofungin analog of the present invention.

### Methods of Treatment

In certain embodiments, the occidiofungin analogs disclosed herein exhibit anti-fungal activity, particularly, against Candida spp, and Saccharomyces spp. Accordingly, certain embodiments of the invention provide methods of administering an occidiofungin analog to a subject in need thereof to treat or prevent an infection, particularly, a fungal infection, more particularly, a yeast infection, and even more particularly, a Candida infection. In specific embodiments, the invention provides methods of treating or preventing an infection caused by a fungus selected from Candida spp., Saccharomyces spp., Trichophyton spp., Rhizopus spp., Mucor spp., Fusarium spp., Aspergillus spp, or Cryptococcus spp., including, for example, Trichophyton mentagrophytes, Trichophyton rubrum, Rhizopus microspores, Mucor circinelloides, Mucor fragilis, Fusarium solani, Fusarium oxysporum, Aspergillus flavus, Aspergillus fumigatus, Candida albicans, Candida glabrata, Candida krusei, Candida auris, Candida parapsilosis, Candida tropicalis, Saccharomyces cerevisiae, or Cryptococcus neoformans.

In certain embodiments, the occidiofungin analogs disclosed herein exhibit anti-parasitic activity, particularly, against Cryptosporidium spp., including, for example Cryptosporidium parvum, Entamoeba histolytica, Giardia duodenalis, or Trichomonas vaginalis. Accordingly, certain embodiments of the invention provide methods of administering an occidiofungin analog to a subject in need thereof to treat or prevent a parasitic infection.

In certain embodiments, the occidiofungin analogs can be used for cancer treatment. In certain embodiments, the occidiofungin analogs exhibit anticancer activity, including, for example, anticancer activity against ovarian cancer, astrocytoma brain cancer (SW1088), and B-cell non-Hodgkin lymphoma. The occidiofungin analog produced can be designed to be conjugated to an antibody targeting a specific tumor or cancer cell line. In certain embodiments, antibodies can be engineered to deliver the enzyme to activate a prodrug occidiofungin analog at the site of tumor or infectious agent.

Cancers suitable for treatment according to the disclosed methods include, but are not limited to: Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-related cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone cancer, Bone tumor, Breast cancer, Brenner tumor, Bronchial tumor, Bronchioloalveolar carcinoma, Brown tumor, Burkitt's lymphoma, Cancer of unknown primary site, Carcinoid tumor, Carcinoma, Carcinoma in situ, Carcinoma of the penis, Carcinoma of unknown primary site, Carcinosarcoma, Castleman disease, Central nervous system embryonal tumor, Cerebellar astrocytoma, Cerebral astrocytoma, Cervical cancer, Cholangiocarcinoma, Chondroma, Chondrosarcoma, Chordoma, Choriocarcinoma, Choroid plexus papilloma, Chronic lymphocytic leukemia, Chronic monocytic leukemia, Chronic myelogenous leukemia, Chronic myeloproliferative disorder, Chronic neutrophilic leukemia, Clear-cell tumor, Colon cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial uterine cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing family of tumors, Ewing sarcoma, Extracranial germ cell tumor, Extragonadal germ cell tumor, Extrahepatic bile duct cancer, Extramammary Paget's disease, Fallopian tube cancer, Fetus in fetu, Fibroma, Fibrosarcoma, Follicular lymphoma, Follicular thyroid cancer, Gallbladder cancer, Ganglioglioma, Ganglioneuroma, Gastric cancer, Gastric lymphoma, Gastrointestinal cancer, Gastrointestinal carcinoid tumor, Gastrointestinal stromal tumor, Germ cell tumor, Germinoma, Gestational choriocarcinoma, Gestational trophoblastic tumor, Giant cell tumor of bone, Glioblastoma multiforme, Glioma, Gliomatosis cerebri, Glomus tumor, Glucagonoma, Gonadoblastoma, Granulosa cell tumor, Hairy cell leukemia, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin's lymphoma, Hypopharyngeal cancer, Hypothalamic glioma, Inflammatory breast cancer, Intraocular melanoma, Islet cell carcinoma, Islet cell tumor, Juvenile myelomonocytic leukemia, Kaposi's sarcoma, Kidney cancer, Klatskin tumor, Krukenberg tumor, Laryngeal cancer, Lentigo maligna melanoma, Leukemia, Lip and oral cavity cancer, Liposarcoma, Lung cancer, Luteoma, Lymphangioma, Lymphangiosarcoma, Lymphoepithelioma, Lymphoid leukemia, Lymphoma, Macroglobulinemia, Malignant fibrous histiocytoma, Malignant fibrous histiocytoma of bone, Malignant glioma, Malignant mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel cell carcinoma, Mesothelioma, Metastatic squamous neck cancer with occult primary, Metastatic urothelial carcinoma, Mixed Müllerian tumor, Monocytic leukemia, Mouth cancer, Mucinous tumor, Multiple endocrine neoplasia syndrome, Multiple myeloma, Mycosis fungoides, Myelodysplasia disease, Myelodysplasia syndromes, Myeloid leukemia, Myeloid sarcoma, Myeloproliferative disease, Myxoma, Nasal cavity cancer, Nasopharyngeal cancer, Nasopharyngeal carcinoma, Neoplasm, Neurinoma, Neuroblastoma, Neurofibroma, Neuroma, Nodular melanoma, Non-Hodgkin's lymphoma, Nonmelanoma skin cancer, Non-small cell lung cancer, Ocular oncology, Oligoastrocytoma, Oligodendroglioma, Oncocytoma, Optic nerve sheath meningioma, Oral cancer, Oropharyngeal cancer, Osteosarcoma, Ovarian cancer, Ovarian epithelial cancer, Ovarian germ cell tumor, Ovarian low malignant potential tumor, Paget's disease of the breast, Pancoast tumor, Pancreatic cancer, Papillary thyroid cancer, Papillomatosis, Paraganglioma, Paranasal sinus cancer, Parathyroid cancer, Penile cancer, Perivascular epithelioid cell tumor, Pharyngeal cancer, Pheochromocytoma, Pineal parenchymal tumor of intermediate differentiation, Pineoblastoma, Pituicytoma, Pituitary adenoma, Pituitary tumor, Plasma cell neoplasm, Pleuropulmonary blastoma, Polyembryoma, precursor T-lymphoblastic lymphoma, Primary central nervous system lymphoma, Primary effusion lymphoma, Primary hepatocellular cancer, Primary liver cancer, Primary peritoneal cancer, Primitive neuroectodermal tumor, Prostate cancer, Pseudomyxoma peritonei, Rectal cancer, Renal cell carcinoma, Respiratory tract carcinoma involving the NUT gene on chromosome 15, Retinoblastoma, Rhabdomyoma, Rhabdomyosarcoma, Richter's transformation, Sacrococcygeal teratoma, Salivary gland cancer, Sarcoma, Schwannomatosis, Sebaceous gland carcinoma, Secondary neoplasm, Seminoma, Serous tumor, Sertoli-Leydig cell tumor, Sex cord-stromal tumor, Sézary syndrome, Signet ring cell carcinoma, Skin cancer, Small blue round cell tumor, Small cell carcinoma, Small cell lung cancer, Small cell lymphoma, Small intestine cancer, Soft tissue sarcoma, Somatostatinoma, Soot wart, Spinal cord tumor, Spinal tumor, Splenic marginal zone lymphoma, Squamous cell carcinoma, Stomach cancer, Superficial spreading melanoma, Supratentorial primitive neuroectodermal tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat cancer, Thymic carcinoma, Thymoma, Thyroid cancer, Transitional cell cancer of renal pelvis and ureter, Transitional cell carcinoma, Urachal cancer, Urethral cancer, Urogenital neoplasm, Uterine sarcoma, Uveal melanoma, Vaginal cancer, Verner-Morrison syndrome, Verrucous carcinoma, Visual pathway glioma, Vulvar cancer, Waldenstrom macroglobulinemia, Warthin's tumor, Wilms' tumor, or any combinations thereof.

In some embodiments, the cancer treated according to the invention is a cancer of the skin, particularly a melanoma; rectum; bladder; cervix; head/neck; thyroid; breast; prostate; uterus; colon; adrenal; hepatocellular; lung, particularly a lung adenoma or lung squamous cancer; or kidney, particularly kidney chromophobe, kidney clear cell or kidney papillary cancer; or a glioma/glioblastoma.

Methods for treating or preventing an infection or cancer can be performed in any subject, such as a mammal, including humans. Such methods comprise administering to a subject in need of such prevention or treatment of an infection or cancer an effective amount of an occidiofungin analog the subject invention. An occidiofungin analog can be administered in the form of a pharmaceutical composition of an occidiofungin analog.

Preferably, an occidiofungin analog is administered parenterally or enterally. Even more preferably, an occidiofungin analog is administered intravaginally, intraperitoneally, subcutaneously or intravenously. The dosage of the effective amount of an occidiofungin analog can vary depending upon the age and condition of each subject to be treated. However, suitable unit dosages typically range from about 0.01 to about 100 mg. For example, a unit dose can be in the range of about 0.2 mg to about 50 mg. Such a unit dose can be administered more than once a day, e.g., two or three times a day.

In certain embodiments, culture media is also provided within the present disclosure. For example, the culture media can comprise or consist of ddH2O supplemented by potassium phosphate dibasic, ammonium chloride, magnesium sulfate heptahydrate, and a carbon source. In preferred embodiments, the carbon source is an amino acid or derivative thereof, citrate, cis-aconitate, D-isocitrate, α-ketoglutarate, succinyl-CoA, succinate, fumarate, malate, oxaloacetate, pyruvate, or acetyl-CoA. In certain embodiments, the concentration of potassium phosphate dibasic is about 1 g/L to about 100 g/L, about 5 g/L to about 50 g/L or about 9 g/L; the concentration of ammonium chloride is about 0.1 g/L to about 10 g/L, about 0.5 g/L to about 5 g/L, or about 1 g/L; the concentration of magnesium sulfate heptahydrate is about 0.01 g/L to about 10 g/L, about 0.1 g/L to about 1 g/L, or about 0.2 g/L; and, the concentration of the carbon source is about 0.1 g/L to about 10 g/L, about 0.5 g/L to about 5 g/L, or about 1 g/L.

Methods of culturing B. contaminans, such as B. contaminans MS14 for the production of the disclosed occidiofungin analogs are also provided. In such methods, the cells can be cultured at a temperature of about 20° C. to about 30° C., for example, about 28° C., in the disclosed media for a period of 6 hours to about 10 days or longer (for example, for a period of about 6 to 24 hours or a period of about 4 days to about 10 days, or a period of about 4, 5, 6, 7, 8, 9, or 10 days).

### Materials and Methods

### Product Isolation

A primary culture was prepared in a 2 L culture flask and consists of 1 L double-distilled water (ddH₂O) supplemented by potassium phosphate dibasic (9 g/L), ammonium chloride (1 g/L), magnesium sulfate heptahydrate (0.2 g/L), and L-asparagine (1 g/L). The pH of the culture was adjusted to 7.0-7.2 before autoclaving for 20 minutes. Secondary cultures were prepared in 2.8 L culture flasks and consisted of 900 ml ddH₂O supplemented with potassium phosphate dibasic (9 g/L), ammonium chloride (1 g/L), magnesium sulfate heptahydrate (0.2 g/L), and L-asparagine (1 g/L). The pH of the culture was adjusted to 7.0-7.2 before autoclaving for 20 minutes. 1 ml of frozen glycerol stock of B. contaminans MS14 was added to the overnight culture and incubated at 28° C. with shaking at 150 RPM. The OD600 of the primary culture was checked in 8 to 15 hours, and 100 ml of the primary culture was added to each flask of the secondary culture at OD600 of 0.65-0.9. Secondary cultures were incubated for 3-7 days at 28° C. with no shaking before extraction. Prior to extraction, the secondary cultures were pooled and heated to 55° C.-60° C. for heat-killing the bacteria before being cooled to 40° C. or below.

A column was prepared by packing with Amberlite XAD1140 resin with at least 2 g of resin per liter of culture being extracted. Prior to packing the column, the resin was submerged in 80% isopropanol and mixed at ambient temperature for 30 minutes before decanting. The resin was then submerged in ddH₂O and mixed at ambient temperature for 15 minutes before decanting, and this water wash was repeated two more times. The resin was then packed into the column following the water washes. Resins can be used for up to 4 extractions before discarding. A peristaltic pump was secured to the column using Masterflex tubing and the flow rate of the pump was measured. The heat-killed culture was pumped through the column for such time that 4-times the total culture volume was circulated through the column. The resin was then rinsed with at least 75 ml of double distilled water per gram of resin. The resin was then rinsed with at least 150 ml of 20% isopropanol (IPA) in ddH₂O containing 0.1% trifluoracetic acid (TFA) for no less than 30 minutes. The resin was then rinsed twice with at least 150 ml of 80% IPA in ddH₂O containing 0.1% TFA for no less than 60 minutes to elute the active compound. The 80% IPA rinses were combined and dried on an evaporator or rotovap to remove the IPA. The water phase was centrifuged at 13,000 g and the resulting pellet was suspended in 35% acetonitrile (ACN) containing 0.1% TFA. The resulting material was further purified by reverse-phase high-performance liquid chromatography (RP-HPLC).

### Product Purification

The product is purified using reverse-phase high-performance liquid chromatography (RP-HPLC) on a SinoChrom ODS-BP 5 μm (10.0 mm×250 mm SN: 3018159x) column. The mobile phases are ACN and ddH₂O both with 0.1% TFA. The gradient develops from 90% ddH₂O to 20% ddH₂O over 30 minutes. Occidiofungin elutes out at around 45%-43% ddH₂O 0.1% TFA. The sample can then be purified further to separate occidiofungin variants. The collected peaks were run on a Supercell ODS2 5_{μm} (4.6 mm×250 mm SN: E3018660) column with ACN and 10 mM triethylamine both with 0.1% formic acid (FA). The gradient develops from 90% 10 mM triethylamine to 60% 10 mM triethylamine and is held for 10 minutes before going down to 20% 10 mM triethylamine. Occidiofungin variants elute during the isocratic hold at 60% 10 mM triethylamine.

### Mass Spectrometry (MS)

Confirmation of product is performed using mass spectrometry (MS) on a ThermoFisher Q Exactive Orbitrap mass spectrometer (ESI-MS) or Bruker Ultraflextreme MALDI-TOF-TOF.

For ESI-MS, compounds were dissolved in 50% ACN with 0.1% FA at a concentration of 1 μg/ml or higher. Samples were directly injected at a rate of 1 μL/min. Protonated masses of the compounds were observed.

For MALDI-TOF MS, a 1:1 ratio of sample to alpha-cyano-4-hydroxycinnamic acid matrix (10 mg/ml) was mixed onto the MALDI-TOF plate and allowed to dry. Samples are analyzed in reflector mode with a low laser power setting (Instrument-ultrafleXtreme TOF/TOF (Bruker corporation, Billerica, A M); Software-flexControl (version 3.4.135.12)/flex analysis (3.4.78.0)) (Laser: Repetition rate-1000 Hz; Number of shots-1000; Beam focus −6; Beam attenuation-70) (Spectrometer: Polarity-Positive voltage polarity; PIE delay −120 ns; Reflector detector voltage −2.625 kV; Lens voltage −7.8 kV; Ion source 1-20 kV; Ion source 2-17.85 kV). Samples were scanned from a range of 650-3000 Da. MS data obtained from MALDI-TOF had a high signal intensity for sodium (+22.98 Da) and potassium (+38.96 Da) adducts. Protonated samples display a lower signal intensity compared to these 2 adducts. Observation of protonated occidiofungin variants with ASN1 instead of BHN1 is not always clear due to weak signal intensity compared to the adducts.

### Nuclear Magnetic Resonance (NMR)

A three to four mg sample of occidiofungin is dissolved in 600 μl of dimethyl sulfoxide (DMSO-d6; Cambridge Isotopes) and the NMR data will be collected as previously reported^{6,7}. The ¹H resonances are assigned according to standard methods⁸ using correlation spectroscopy (COSY), total correlation spectroscopy (TOCSY), and nuclear Overhauser effect spectroscopy (NOESY) experiments. Rotating-frame Overhauser enhancement spectroscopy (ROESY) and ¹³C-heteronuclear single quantum correlation (HSQC) experiments are used to clarify some areas of ambiguity in the TOCSY and NOESY spectra. The spectral sweep width for the TOCSY, NOESY and ROESY is typically 11.35 ppm in both dimensions. The spectral sweep widths for HSQC are typically 11.35 ppm in the proton dimensions and 100 and 150 ppm in the carbon dimension. All two-dimensional (2D) data are collected with 2,048 complex points in the acquisition dimension and 256 complex points for the indirect dimensions, except for the HSQC, which is collected with 2,048 and 128 complex points in the direct and indirect dimensions, respectively. Data are processed with nmrPipe⁹ and analyzed with the interactive computer program NMR View¹⁰.

### Overlay Assays

Yeast extract peptone dextrose (YPD) media plates (20 g/L peptone, 20 g/L dextrose, 10 g/L yeast extract 15 g/L agar) are overlaid with YPD soft media agar (20 g/L peptone, 20 g/L dextrose, 10 g/L yeast extract 7.5 g/L agar) containing S. cerevisiae DGY6 (OD600=0.15 followed by a 25-fold dilution) and left to harden. Compounds in solution are then spotted on the plate and left to dry. Equal volumes and quantity are spotted onto the plate for comparison of different compounds. The plate is incubated at 35° C. for 24 hours. The plate is then observed for zones of growth inhibition.

### Minimum Inhibitory Concentration (MIC) Assays

MIC assays were performed according to a modified CLSI M27-A3 method with YPD growth media or YPD growth media supplemented with human or mouse serum, to a solution with 20% serum and 80% growth media (Serum MIC), in a 96-well plate. The plate is incubated at 35° C. for 24 hours, and MIC values are determined by examining for the presence of fungi in individual wells on the plate.

### Semi-Synthetic Analog Production

Analog productions on variants with ASP7 are performed with 1-ethyl-3-(3-dimethylaminopropyl) carbodiimide hydrochloride (EDC) as condensation reagent to produce various amides at the carboxyl group on ASP7 (FIG. 5A). OCF-I and OCF-J are incubated with a 10× excess molar ratio of the desired amine, EDC, and 1-Hydroxybenzotriazole (HOBt) in N—N-dimethylformamide (DMF) or dimethyl sulfoxide (DMSO) at room temperature for 16 hours or longer, and in the absence of light. Longer incubation time or incubating at 35° C. has shown increased efficiency for some of the amines used in the reaction, particularly the larger amines. Incubating for 4 hours or less does not result in the reaction occurring using the described conditions. Primary amines have been successfully used in this reaction with carbon chain length up to 12 carbons (dodecylamine). Two primary amines with a terminal alkyne group, but-3-yn-1-amine and propargylamine, has also been successfully used in this reaction. The reactions can also be performed with GLU7 variants to produce amides at the carboxyl group on GLU7 (FIG. 5B). The reaction conditions are performed in the same manner as the ASP7 variants.

Direct EDC condensation could not be achieved using 3-amino-1-propanol. This may be due to the alcohol group present in the compound. The alcohol group of 3-amino-1-propanol was protected with tert-Butyldimethylsilyl (TBS) by adding 1 molar equivalence of 3-amino-1-propanol, triethylamine and tert-Butyldimethylsilyl chloride (TBS-Cl) in dichloromethane (DCM). The reaction mixture is mixed and incubated at room temperature for at least 4 hours. The oily layer produced after incubation contains the protected 3-amino-1-propanol, and is used as the amine in the EDC condensation reaction. TBS protection of 3-aminopropyl-dihydrogen-phosphate was also necessary for production of the phosphate prodrug designs OCF-S21 and OCF-S22. 1 molar equivalence of 3-aminopropyl-dihydrogen-phosphate, triethylamine and TBS-Cl is incubated in DCM. The reaction mixture is mixed and equal volume of 35% acetonitrile 0.1% trifluoroacetic acid is added, and is then incubated at room temperature for no less than 4 hours. The top aqueous layer contains the protected 3-aminopropyl-dihydrogen-phosphate and is subsequently used in the EDC condensation reaction.

To produce the methyl and methyl succinate prodrugs, Boc-3-amino-1-propanol was first conjugated to the methyl or benzyl succinate. 1 molar equivalence of 3-amino-1-propanol, N,N′-Dicyclohexylcarbodiimide (DCC), methyl succinate/benzyl succinate and 0.1 molar equivalence of 4-dimethylaminopyridine (DMAP) was added to anhydrous dimethylformamide (DMF). The reaction was stirred at room temperature for at least 12 hours. Dicyclohexyl urea by-product is seen as a white precipitate. The solution is filtered through a 2 micron filter paper to remove the by-product. Saturated ammonium chloride was added to the reaction mixture and the reaction mixture was extracted with ethyl acetate twice. The combined ethyl acetate extracts were dried using anhydrous magnesium sulphate. The ethyl acetate is then filtered through a 2 micron filter paper and dried on a rotary evaporator. The residue left behind is the product and is brought up in a 20% trifluoracetic acid 80% dichloromethane solution and left at room temperature for 24 hours. 1 molar excess of saturated sodium bicarbonate is then added to neutralize the TFA. The reaction solution is extracted twice with ethyl acetate, and the combined ethyl acetate extracts are dried with anhydrous magnesium sulphate before filtering through a 2 micron filter paper. The ethyl acetate is then dried on a rotary evaporator and brought up in ethanol as a stock solution and stored at −20° C. The final product was confirmed by mass spectrometry. The synthesized EDC condensation reactions were quenched by diluting to 1 ml using 35% ACN 0.1% TFA and ran on a Supercell ODS2 5 μm (4.6 mm×250 mm SN: E3018660) column. Peaks are collected and analyzed MALDI-TOF or ESI-MS to confirm the expected product.

### OCF-S21/OCF-S22 Phosphate Prodrug Activation

The removal of the phosphate group of OCF-S21/OCF-S22 in vitro was achieved by using calf intestinal alkaline phosphatase (Quick CIP; New England Biolabs). For the overlay assay, 5 μg of OCF-S21/OCF-S22 (around 5000 μmol) was incubated with 0.25 μl of the alkaline phosphatase, 0.5 μl of the rCutSmart™ buffer (1×), and 3.25 μl ddH₂O. The reaction mixture was incubated at 37° C. for at least 90 minutes. Remove of the phosphate group to produce OCF-S19/OCF-S20 was confirmed by MALDI-TOF MS, and by performing an overlay assay as described previously.

The minimum inhibitory concentration assay was performed according to a modified CLSI M27-A3 method with YPD growth media. Each well was supplemented with 10 μl of the alkaline phosphatase, 2 μl of the rCutSmart™ buffer (10×) such that the final volume of each well totaled 200 μl. A negative control containing 10 μl of the alkaline phosphatase and 2 μl of the rCutSmart™ buffer (10×) with no OCF-S21/OCF-S22 showed no inhibition of fungal growth.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1—Aspartic Acid 7 Occidiofungin

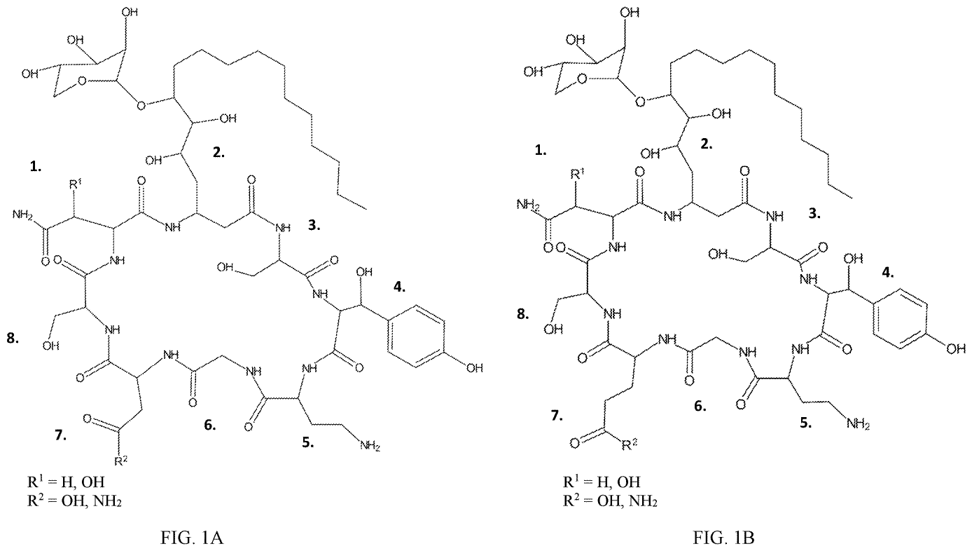

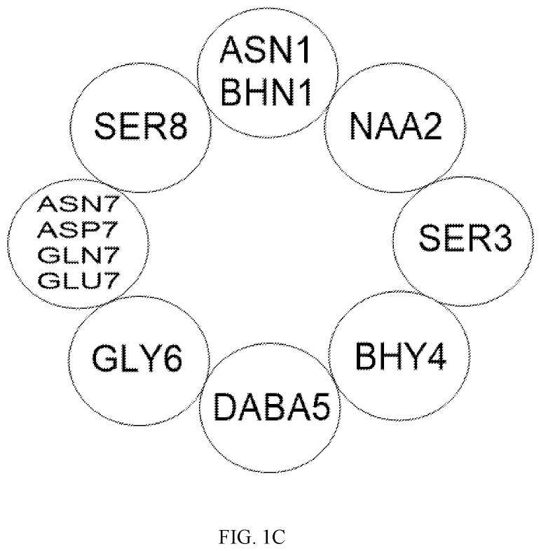

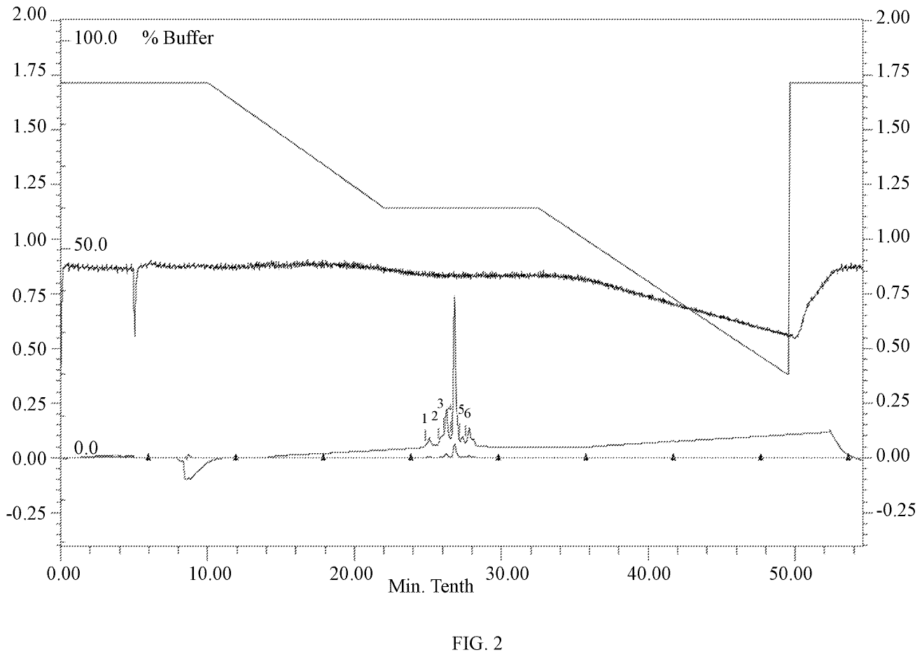

Occidiofungin is extracted from B. contaminans MS14 culture as described in detail in the Methods section. Occidiofungin is a cyclic glycopeptide produced non-ribosomally and is composed of 8 amino acids (FIG. 1). Briefly, B. contaminans MS14 culture is pumped through Amberlite XAD1180 resin which collects occidiofungin as well as other materials. 80% Isopropanol (IPA) with 0.1% trifluoracetic acid (TFA) is used to elute out occidiofungin from the resin. The 80% IPA 0.1% TFA is dried down and brought up in solution with 35% acetonitrile (ACN) 0.1% TFA. The solution is purified by reverse phase-high performance liquid chromatography (RP-HPLC) using ACN and double distilled water (ddH₂O) both containing 0.1% TFA. Occidiofungin elutes as 2 peaks between 45%-43% ddH₂O 0.1% TFA. The peaks are collected and further purified by running with ACN and 10 mM triethylamine (0.1% FA) mobile phase. Using this approach, the separation of multiple occidiofungin variants can be accomplished (FIG. 2). Peak 2 and 3 on the chromatogram contain OCF variants with asparagine (OCF-A and OCF-B) or glutamine (OCF-K and OCF-L), which NMR structural analyses has shown to be at residue 7. Peaks 4 and 5 contain OCF variants with aspartic acid (OCF-I and OCF-J) or glutamic acid (OCF-M and OCF-N) on residue 7.

TABLE 1
Indicates the variants eluting out at the demarcated peaks in FIG. 2.

| Peak no. | Occidiofungin variants |
|---|---|
| 1 | Unassigned variant |
| 2 | OCF-A, OCF-B, OCF-L-K, OCF-L |
| 3 | OCF-A, OCF-B, OCF-L-K, OCF-L |
| 4 | OCF-I, OCF-J, OCF-M, OCF-N |
| 5 | OCF-I, OCF-J, OCF-M, OCF-N |
| 6 | Unassigned variant |

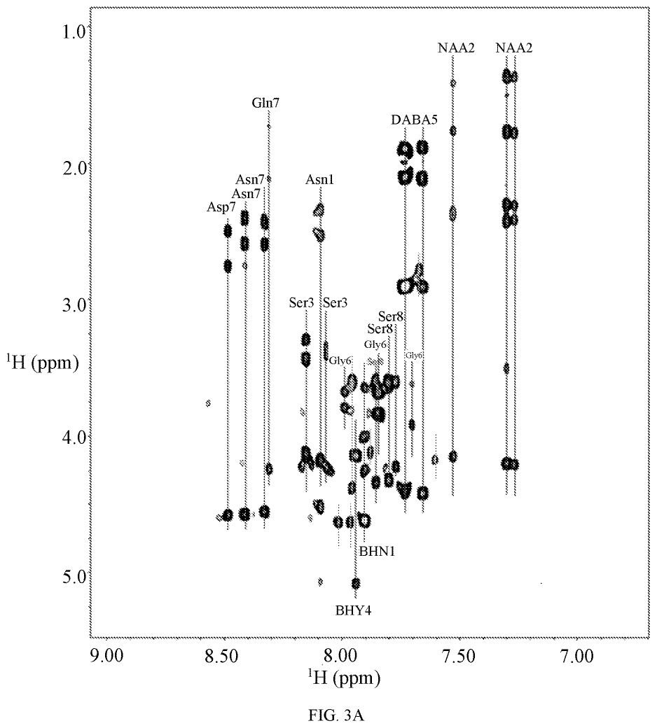

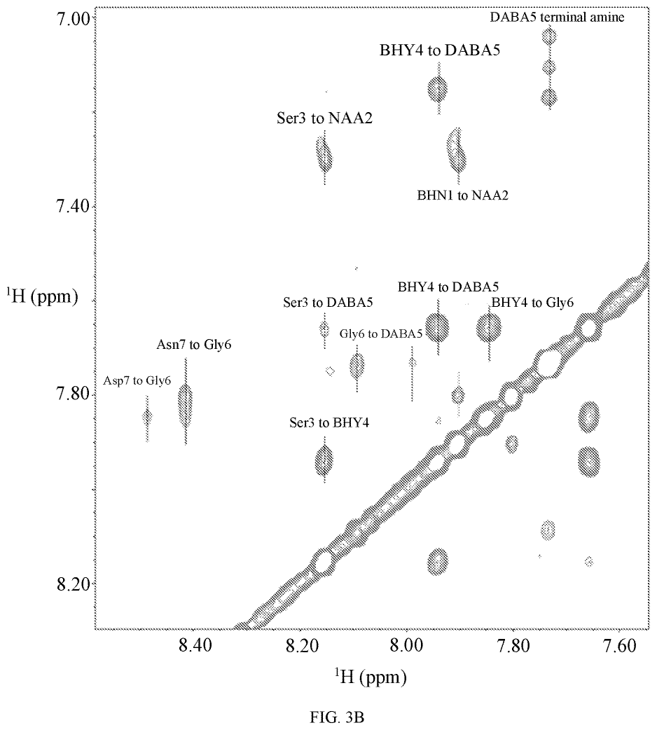

High-resolution mass spectrometry, which provides accurate predictions of elemental composition, shows natural occidiofungin variants with ASP, GLN, and GLU instead of ASN (FIGS. 3A-3B, Table 2). NMR data obtained from 2 samples were used to confirm the presence of ASP at position 7 (FIGS. 3A-3B). A sample containing a mixture of OCF-A/OCF-B and OCF-I/OCF-J (FIGS. 3A-3B) showed the presence of a spin system corresponding to ASP7 in the TOCSY fingerprint region. The fingerprint region is useful in the assignment of amino acid identity, and the spin system with the H^{N} shift of 8.48 ppm has been assigned as ASN7 in previous studies⁷ (FIG. 3A). ASN and ASP have similar proton shifts, but ASP does not have the proton shifts corresponding to the delta protons on the terminal amide side chain found in ASN residue. This new spin system did not have proton shifts in NOESY corresponding to the beta protons to the delta protons of an ASN, which was used to assign the new spin system as an ASP (FIG. 3B). NOEs are seen from H^{α} and H^{β} of the ASP spin system to H^{N} of SER8, and from H^{α} of Gly6 to the H^{N} of the ASP spin system. These NOEs are also seen in the ASN7 spin systems with H^{N} of 8.41 and 8.32 ppm, and this helps to confirm the ASP residue to be in the seventh position. HRMS data has identified OCF-L and OCF-N which have GLN7 and GLU7 residues (FIGS. 3A-3B, Table 2). The TOCSY data revealed a new spin system that has been assigned as GLN7 with an H^{N} shift of 8.30. The alpha, beta, epsilon, and gamma protons are seen for this spin system which corresponds to GLN. NOEs are seen from H^{α} and H^{β} of the GLN7 spin system to H^{N} of SER8.

TABLE 2
Proton shift for a sample containing a mixture of OCF-A/OCF-B and
OCF-I/OCF-J. The newly assigned ASP7 spin system is shown, and
a new spin system assigned as GLN7 is also shown. The chemical
shift values correspond to the NMR data seen in FIGS. 3A-3B.

| Amino | | | | |
|---|---|---|---|---|
| Acid | H^{N} | H^{α} | H^{β} | Other protons |
| ASN1 | 8.09 | 4.52 | 2.52, 2.35 | γ-NH2: 7.26, 6.86 |
| BHN1 | 7.90 | 4.62 | 4.01 | γ-NH2: 7.30, 6.93 β-OH: |
| | | | | 5.70 |
| NAA2 | 7.53 | | | C2—CH2: 2.32, 2.40 C3—CH2: |
| | | | | 4.15, C4—CH2: 1.76, |
| | | | | 1.35, C5—CH: 3.51, C5—OH: |
| | | | | 4.17, C6—CH: 3.05, C6—OH: |
| | | | | 4.08, C7—CH: 3.75, C8—CH: |
| | | | | 1.36, C9—C17—CH2: |
| | | | | 1.31, C18—CH3: 0.86 |
| NAA2 | 7.30 | | | C2—CH2: 2.32, 2.40 C3—CH2: |
| | | | | 4.15, C4—CH2: 1.76, |
| | | | | 1.35, C5—CH: 3.51, C5—OH: |
| | | | | 4.17, C6—CH: 3.05, C6—OH: |
| | | | | 4.08, C7—CH: 3.75, C8—CH: |
| | | | | 1.36, C9—C17—CH2: |
| | | | | 1.31, C18—CH3: 0.86 |
| NAA2 | 7.28 | | | C2—CH2: 2.32, 2.40 C3—CH2: |
| | | | | 4.15, C4—CH2: 1.76, |
| | | | | 1.35, C5—CH: 3.51, C5—OH: |
| | | | | 4.17, C6—CH: 3.05, C6—OH: |
| | | | | 4.08, C7—CH: 3.75, C8—CH: |
| | | | | 1.36, C9—C17—CH2: |
| | | | | 1.31, C18—CH3: 0.86 |
| SER3 | 8.15 | 4.14 | 3.42, 3.29 | β-OH: 4.96 |
| SER3 | 8.07 | 4.22 | 3.42, 3.34 | β-OH: 4.97 |
| BHY4 | 7.94 | 4.14 | 5.08 | β-OH: 5.75, OH: 9.33, |
| | | | | C2&C6—CH: 7.15, |
| | | | | C3&C5—CH: 6.67 |
| DABA5 | 7.66 | 4.41 | 2.10, 1.87 | γ-H: 2.90, NH2: 7.73 |
| GLY6 | 7.98 | 3.79, 3.68 | | |
| GLY6 | 7.85 | 3.84, 3.68 | | |
| GLY6 | 7.70 | 3.92, 3.61 | | |
| ASN7 | 8.41 | 4.57 | 2.59, 2.41 | γ-NH2: 7.39, 6.93 |
| ASN7 | 8.32 | 4.55 | 2.59, 2.42 | γ-NH2: 7.38, 6.94 |
| ASP7 | 8.48 | 4.58 | 2.75, 2.50 | |
| GLN7 | 8.30 | 4.25 | 2.11, 1.88 | ε-NH2: 7.15.14, 6.67, |
| | | | | C3—CH2: 1.73 |
| SER8 | 7.85 | 4.33 | 3.59 | β-OH: 4.83 |
| SER8 | 7.80 | 4.34 | 3.61 | β-OH: 4.82 |
| SER8 | 7.77 | 4.22 | 3.60 | β-OH: 4.82 |
| Xylose | | | | C1—CH2: 4.18, C2—CH: 2.99, |
| | | | | C2—OH: 4.94 C3—CH: 3:10, |
| | | | | C3—OH: 4.94, C4—CH: 3.30, |
| | | | | C4—OH: 4.93, C5—CH2: |
| | | | | 3.72, 3.06 |

A GLY6 spin system with an H^{N} shift of 7.71 has also been assigned (FIG. 3A) which has an NOE from the H^{α} to the H^{N} of GLN7, which supports the position of the GLN at position 7. The low concentration of the GLN7 and GLU7 variants makes identification by NMR difficult due to the low intensity of the proton shifts and overlapping of proton shifts of more abundant variants. The GLU7 spin system has not yet been assigned, but HRMS data and chemical modifications support the presence of the GLU7 variant. TOCSY and NOESY analysis was also used to assign spin systems corresponding to previously characterized amino acids in occidiofungin^{7,8}. The TOCSY data has a few unassigned spin systems which likely correspond to contaminants from solvent, purification or very minor occidiofungin variants. HSQC data was used to help assign the proton shifts for the NAA2 side chain and the xylose sugar.

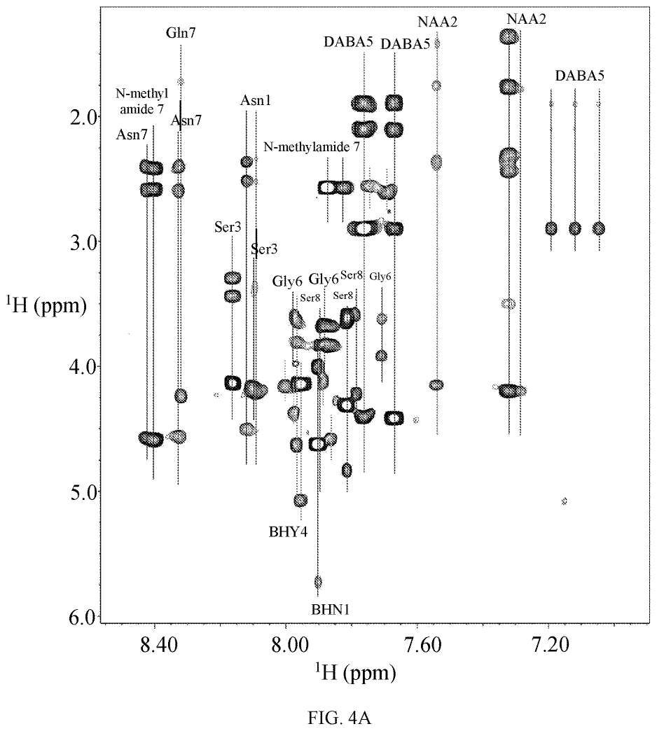

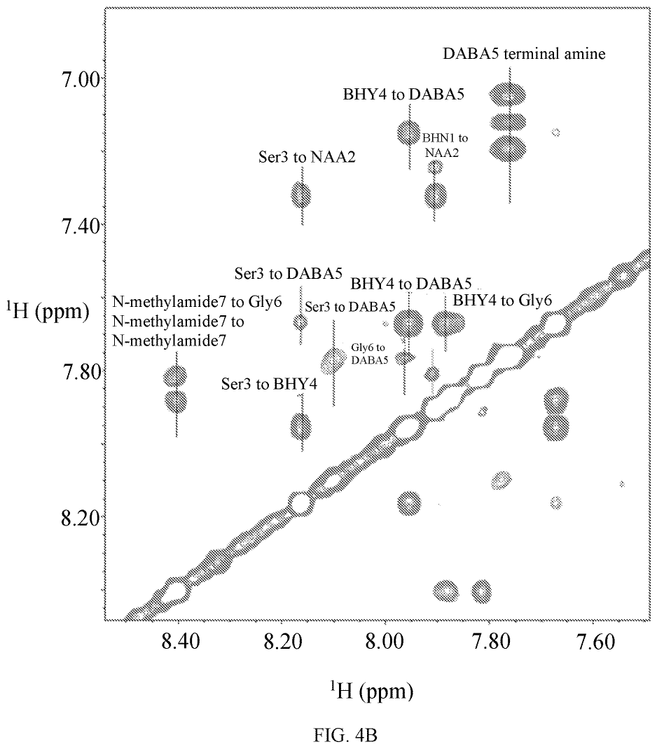

Occidiofungin (from the same batch used in the NMR analysis) was subjected to EDC mediated condensation with methylamine to produce OCF-S3 and OCF-S4 (FIGS. 4A-4B, Table 4). TOCSY of the fingerprint region showed the disappearance of the ASP7 spin system (FIG. 4A, Table 3). EDC condensation with methylamine is the most efficient of the reactions, and MS data shows that all OCF-I/OCF-J is converted to OCF-S3/OCFS4. This corresponds with the disappearance of the ASP7 spin system. A new spin system with an H^{N} of 8.40 is present and slightly overlaps another spin system which is assigned as ASN7 (FIG. 4A, Table 3). Both ASN7 spin systems and the GLN7 spin system are seen in the TOCSY meaning that they are not altered by the EDC condensation reaction. The new spin system is assigned as the ASP7 modified to have a terminal N-methylamide. N-methylamide on ASP7 has similar proton shifts as ASP7 for H^{α} and H^{β} as this part of the amino acid is unaltered. NOEs are seen from H^{α} and H^{β} of the N-methylamide7 spin system to H^{N} of SER8, and from H^{α} of Gly6 to the H^{N} of the ASP spin system (FIG. 4B). NOEs are seen from HP to γ-NH and C6-CH3 terminal methyl group. There are 2 spin systems for the terminal methylamide with γ-NH proton shifts of 7.87 and 7.82, and C6-CH₃ proton shifts of 2.56 for both. HSQC NMR shows that the C13 shift of CH3 on the amide Nitrogen is 26.11. TOCSY data also shows three new spin systems for the terminal amine of DABA5 (FIG. 4A). This suggests that the DABA5 may be involved in the production of a side product in the EDC condensation reactions and this reaction need to be further optimized. No significant alterations were observed for the spin systems of other amino acids in the compound. HSQC data was used to help assign the proton shifts for the NAA2 side chain and the xylose sugar.

TABLE 3
Proton shift for a sample of OCF-S3 and OCF-S4 produced from EDC coupling
of OCF-I and OCF-J (FIG. 5A). The ASP7 spin system is no longer present.
A spin system assigned as N-methylamide ASP7 is present, where the ASP7 is
modified through EDC condensation with methylamine. A proton shift at 2.56
ppm is seen corresponding to the terminal carbon in the side chain is seen.
The chemical shift values correspond to the NMR data seen in FIGS. 4A-4B.

| Amino | | | | |
|---|---|---|---|---|
| Acid | H^{N} | H^{α} | H^{β} | Other protons |
| ASN1 | 8.12 | 4.50 | 2.51, 2.37 | γ-NH2: 7.28, 6.87 |
| ASN1 | 8.08 | 4.53 | 2.51, 2.34 | γ-NH2: 7.28, 6.87 |
| BHN1 | 7.90 | 4.62 | 4.00 | γ-NH2: 7.32, 6.94 β-OH: |
| | | | | 5.72 |
| NAA2 | 7.54 | | | C2—CH2: 2.32, C3—CH2: |
| | | | | 4.14, C4—CH2: 1.77, 1.37, |
| | | | | C5—CH: 3.50, C5—OH: 4.18, |
| | | | | C6—CH: 3.06, C6—OH: 4.09, |
| | | | | C7—CH: 3.75, C8—CH: 1.54, |
| | | | | C9—C17—CH2: 1.27, |
| | | | | C18—CH3: 0.85 |
| NAA2 | 7.32 | | | C2—CH2: 2.32, C3—CH2: |
| | | | | 4.14, C4—CH2: 1.77, 1.37, |
| | | | | C5—CH: 3.50, C5—OH: 4.18, |
| | | | | C6—CH: 3.06, C6—OH: 4.09, |
| | | | | C7—CH: 3.75, C8—CH: 1.54, |
| | | | | C9—C17—CH2: 1.27, |
| | | | | C18—CH3: 0.85 |
| NAA2 | 7.28 | | | C2—CH2: 2.32, C3—CH2: |
| | | | | 4.14, C4—CH2: 1.77, 1.37, |
| | | | | C5—CH: 3.50, C5—OH: 4.18, |
| | | | | C6—CH: 3.06, C6—OH: 4.09, |
| | | | | C7—CH: 3.75, C8—CH: 1.54, |
| | | | | C9—C17—CH2: 1.27, |
| | | | | C18—CH3: 0.85 |
| SER3 | 8.16 | 4.13 | 3.43, 3.30 | β-OH: 4.98 |
| SER3 | 8.09 | 4.19 | 3.46, 3.30 | β-OH: 4.99 |
| BHY4 | 7.95 | 4.14 | 5.06 | β-OH: 5.76, OH - 9.33, |
| | | | | C2&C6: CH - 7.15, |
| | | | | C3&C5: CH - 6.67 |
| DABA5 | 7.67 | 4.42 | 2.10, 1.90 | γ-H: 2.90, NH2: 7.75 |
| GLY6 | 7.98 | 3.80, 3.60 | | |
| GLY6 | 7.88 | 3.81, 3.68 | | |
| GLY6 | 7.70 | 3.91, 3.62 | | |
| ASN7 | 8.42 | 4.58 | 2.59, 2.40 | γ-NH2: 7.40, 6.94 |
| ASN7 | 8.32 | 4.56 | 2.58, 2.40 | γ-NH2: 7.40, 6.94 |
| GLN7 | 8.31 | 4.24 | 2.09, 1.89 | ε-NH2: 7.14, 7.04, C3—CH2; |
| | | | | 1.73 |
| N-methylamide | 8.40 | 4.58 | 2.58, 2.41 | γ-NH: 7.87, 7.82, N—CH3 |
| ASP7 | | | | (amide methyl): 2.56 |
| SER8 | 7.90 | 4.62 | 3.82 | β-OH: 4.83 |
| SER8 | 7.81 | 4.31 | 3.62 | β-OH: 4.83 |
| Xylose | | | | C1—CH2: 4.19, C2—CH: 2.99. |
| | | | | C2—OH: 4.96, C3—CH: 3:11, |
| | | | | C3—OH: 4.96, C4—CH: 3.30, |
| | | | | C4—OH: 4.98, C5—CH2: 3.72, |
| | | | | 3.06 |

High-resolution mass spectrometry analysis of occidiofungin reveals masses that also correspond to OCF-K/OCF-L containing glutamine (GLN) or OCF-M/OCF-N (GLU) as residue 7 (Table 4). OCF-K/OCF-L coelutes out with OCF-A/OCF-B and OCF-M/OCF-N co-elutes out with OCF-I/OCF-J during HPLC purification. Glutamine and glutamic acid have an additional carbon in the side chain compared to asparagine and aspartic acid respectively. Given the structural similarities between the amino acids in the 7^{th} residue for the variants, the non-ribosomal peptide synthetase responsible for the addition of this residue (OcfD) is likely capable of accommodating the structurally related amino acids. Non-ribosomal peptide synthetases may display nonspecific, but preferential, binding for a particular amino acid¹⁵. OcfD may preferentially bind asparagine but may also bind aspartic acid, glutamine, or glutamic acid. The existence of ASP7, GLN7, and GLU7 variants was unknown during the NMR analysis of thioesterase ocfN mutants¹⁶. OcfN was shown to recognize ASNI to produce OCF-A, but could also possibly recognize ASP7, GLN7, or GLU7 to produce other variants.

TABLE 4
Native occidiofungin variants identified by HRMS, with their
description and predicted elemental composition. The description
predicts the structure of the variant and is supported by
chemical modifications and NMR as well as HRMS.

| | | Elemental Composition |
|---|---|---|
| | | (M + H) Mass |
| Variant | Description | (m/z HRMS) |
| OCF-A | Asn at position 1 (SEQ ID | C₅₂H₈₆O₂₁N₁₁ (1200.59942) |
| | NO: 1) | |
| OCF-B | β-hydroxy ASN at position | C₅₂H₈₆O₂₂N₁₁ (1216.59434) |
| | 1 (SEQ ID NO: 2) | |
| OCF-C | Chlorination of β-hydroxy Try | Not observed |
| | on the OCF-A (SEQ ID NO: 3) | |
| OCF-D | Chlorination of β-hydroxy Try | Not observed |
| | on the OCF-B (SEQ ID NO: 4) | |
| OCF-E | Xylose Free variant of the | Not observed |
| | OCF-A (SEQ ID NO: 5) | |
| OCF-F | Xylose Free variant of the | C₄₇H₇₇O₁₈N₁₁ (1083.55208) |
| | OCF-B (SEQ ID NO: 6) | |
| OCF-G | Xylose Free variant of the | Not observed |
| | OCF-I (SEQ ID NO: 7) | |
| OCF-H | Xylose Free variant of the | C₄₇H₇₆O₁₉N₁₀ (1084.53609) |
| | OCF-J (SEQ ID NO: 8) | |
| OCF-I | Aspartic Acid (Asp) at position | C₅₂H₈₅O₂₂N₁₀ (1201.583995) |
| | 7 instead of Asn observed in | |
| | OCF-A (SEQ ID NO: 9) | |
| OCF-J | Aspartic Acid (Asp) at position | C₅₂H₈₅O₂₃N₁₀ (1217.57835) |
| | 7 instead of Asn observed in | |
| | OCF-B (SEQ ID NO: 10) | |
| OCF-K | Glutamine (Gln) at position | Not observed |
| | 7 instead of Asn observed in | |
| | OCF-A (SEQ ID NO: 11) | |
| OCF-L | Glutamine (Gln) at position | C₅₃H₈₈O₂₂N₁₁ (1230.60999) |
| | 7 instead of Asn observed in | |
| | OCF-B (SEQ ID NO: 12) | |
| OCF-M | Glutamic acid (Glu) at position | Not observed |
| | 7 instead of Asn observed in | |
| | OCF-A (SEQ ID NO: 13) | |
| OCF-N | Glutamic acid (Glu) at position | C₅₃H₈₇O₂₃N₁₀ (1231.59456) |
| | 7 instead of Asn observed in | |
| | OCF-B (SEQ ID NO: 14 | |

TABLE 5
Semi-synthetic occidiofungin analogs produced using OCF-I and OCF-J variants with
their description, theoretical mass and observed mass from MALDI-TOF or ESI-MS.
The theoretical mass corresponds to the predicted protonated mass. Some of the
theoretical masses are not observed due to usage of MALDI-TOF MS, where sodium
and potassium adducts have a higher signal intensity than the protonated compound.
The semi-synthetic analogs can also be produced through EDC coupling with OCF-M
and OCF-N, containing glutamic acid at position 7, are not listed.

| | | Theoretical | Ob- |
|---|---|---|---|
| | | protonated | served |
| Variant | Description | mass | mass |
| OCF-S1 | OCF-I EDC condensation with | 1200.599 | 1200.577 |
| | ammonia | | |
| | Formula (I) R1 = H, R2 = H, R3 = H | | |
| OCF-S2 | OCF-J EDC condensation with | 1216.594 | 1216.579 |
| | ammonia | | |
| | Formula (I) R1 = OH, R2 = H, R3 = | | |
| | H | | |
| OCF-S3 | N-methyl amide at position | 1214.319 | 1214.588 |
| | 7 instead of Asp observed in OCF-I | | |
| | Formula (I) R1 = H R2 = H, R3 = | | |
| | CH₃ | | |
| OCF-S4 | N-methylamide at position | 1230.318 | 1230.580 |
| | 7 instead of Asp observed at OCF-J | | |
| | Formula (I) R1 = OH R2 = H, R3 = | | |
| | CH₃ | | |
| OCF-S5 | N-dimethylamide at position | 1228.345 | Not seen |
| | 7 instead of Asp observed in OCF-I | | |
| | Formula (I) R1 = H R2 = CH2, R3 = | | |
| | CH₂ | | |
| OCF-S6 | N-dimethylamide at position | 1244.345 | 1244.599 |
| | 7 instead of Asp observed in OCF-J | | |
| | Formula (I) R1 = OH R2 = CH2, R3 = | | |
| | CH₂ | | |
| OCF-S7 | N-ethylamide at position | 1228.345 | 1228. 635 |
| | 7 instead of Asp observed in OCF-I | | |
| | Formula (I) R1 = H R2 = H, R3 = | | |
| | CH₂CH₃ | | |
| OCF-S8 | N-ethylamide at position | 1244.345 | 1244.629 |
| | 7 instead of Asp observed in OCF-J | | |
| | Formula (I) R1 = OH R2 = H, R3 = | | |
| | CH₂CH₃ | | |
| OCF-S9 | N-propylamide at position | 1242.372 | 1242.620 |
| | 7 instead of Asp observed in OCF-I | | |
| | Formula (I) R1 = H R2 = H, R3 = | | |
| | CH₂CH₂CH₃ | | |
| OCF-S10 | N-propylamide at position | 1258.371 | 1258.614 |
| | 7 instead of Asp observed in OCF-J | | |
| | Formula (I) R1 = OH R2 = H, R3 = | | |
| | CH₂CH₂CH₃ | | |
| OCF-S11 | N-undecylamide at position | 1354.584 | Not |
| | 7 instead of Asp observed in OCF-I | | observed |
| | Formula (I) R1 = H R2 = H, R3 = | | |
| | (CH₂₎₁₀CH₃ | | |
| OCF-S12 | N-undecylamide at position | 1370.584 | 1370.738 |
| | 7 instead of Asp observed in OCF-J | | |
| | Formula (I) R1 = OH R2 = H, R3 = | | |
| | (CH₂₎₁₀CH₃ | | |
| OCF- S13 | N-dodecylamide at position | 1368.611 | 1368.811 |
| | 7 instead of Asp observed in OCF-I | | |
| | Formula (I) R1 = H R2 = H, R3 = | | |
| | (CH₂₎₁₁CH₃ | | |
| OCF- S14 | N-dodecylamide at position | 1384.610 | 1384.814 |
| | 7 instead of Asp observed in OCF-J | | |
| | Formula (I) R1 = OH R2 = H, R3 = | | |
| | (CH₂₎₁₁CH₃ | | |
| OCF-S15 | Unassigned dehydrated OCF-I | 1183.568 | Not |
| | | | observed |
| OCF-S16 | Unassigned dehydrated OCF-J | 1199.563 | 1199.391 |
| OCF-S17 | N-TBS-3-hydroxy-propylamide at | 1372.632 | Not |
| | position 7 instead of Asp observed in | | observed |
| | OCF-I | | |
| | Formula (I) R1 = H R2 = H, R3 = | | |
| | CH₂CH₂ CH₂OSi(CH₃)₂C(CH₃)₃ | | |
| OCF-S18 | N-TBS-3-hydroxy-propylamide at | 1388.632 | Not |
| | position 7 instead of Asp observed in | | observed |
| | OCF-J | | |
| | Formula (I) R1 = OH R2 = H, R3 = | | |
| | CH₂CH₂ CH₂OSi(CH₃)₂C(CH₃)₃ | | |
| OCF- S19 | N-3-hydroxy-propylamide at position | 1258.371 | Not |
| | 7 instead of Asp observed in OCF-I | | observed |
| | Formula (I) R1 = H R2 = H, R3 = | | |
| | CH₂CH₂ CH₂OH | | |
| OCF-S20 | N-3-hydroxy-propylamide at position | 1274.371 | 1274.403 |
| | 7 instead of Asp observed in OCF-J | | |
| | Formula (I) R1 = OH R2 = H, R3 = | | |
| | CH₂CH₂ CH₂OH | | |
| OCF-S21 | N-3-amino-propyl-dihydrogen- | 1338.607 | 1338.541 |
| | phosphate at position 7 instead of Asp | | |
| | observed in OCF-I | | |
| | Formula (I) R1 = H R2 = H, R3 = | | |
| | CH₂CH₂ CH₂OPO₃H₂ | | |
| OCF-S22 | N-3-amino-propyl-dihydrogen- | 1354.602 | 1354.547 |
| | phosphate at position 7 instead of Asp | | |
| | observed in OCF-J | | |
| | Formula (I) R1 = OH R2 = H, R3 = | | |
| | CH₂CH₂ CH₂OPO₃H₂ | | |
| OCF-S23 | N-3-amino-propyl-methyl-succinate | 1372.470 | Not |
| | ester at position 7 instead of Asp | | observed |
| | observed in OCF-I | | |
| | Formula (I) R1 = H R2 = H, R3 = | | |
| | CH₂CH₂ CH₂OCOCH₂CH₂CO₂CH₃ | | |
| OCF-S24 | N-3-amino-propyl-methyl-succinate | 1388.470 | Not |
| | ester at position 7 instead of Asp | | observed |
| | observed in OCF-J | | |
| | Formula (I) R1 = OH R2 = H, R3 = | | |
| | CH₂CH₂ CH₂OCOCH₂CH₂CO₂CH₃ | | |
| OCF-S25 | N-but-3-yn-1-amine at position 7 | 1252.631 | 1252.632 |
| | instead of Asp observed in OCF-I | | |
| | Formula (I) R1 = H R2 = H, R3 = | | |
| | CH₂CH₂ CCH | | |
| OCF-S26 | N-but-3-yn-1-amine at position 7 | 1268.625 | 1268.644 |
| | instead of Asp observed in OCF-I | | |
| | Formula (I) R1 = OH R2 = H, R3 = | | |
| | CH₂CH₂ CCH | | |

### Example 2—Aspartic Acid 7 Semi-Synthetic Analogs

EDC condensation is performed by incubating OCF-I and OCF-J with 10-molar excess of EDC, hydroxybenzotriazole (HOBt) and the amine of choice in dimethylformamide (DMF) or dimethyl sulfoxide (DMSO) (FIG. 5A). EDC condensation can also be performed with OCF-M and OCF-N variant (FIG. 5B). The reaction mixture is incubated and is purified by RP-HPLC as described in the methods section.

The modification itself can result in a change in retention time during RP-HPLC. Multiple primary amines were successful in the condensation reaction, and one secondary amine (dimethylamine) was also successful (FIG. 5A). Benzylamine condensation was not successful, possibly due to steric hindrance caused by the benzene ring, even when the reaction was performed at elevated temperature and extended time. Restoration of OCF-A and OCF-B from OCF-I and OCF-J is also possible by EDC mediated condensation with ammonia. Larger amines used in the condensation increase the hydrophobicity of the analog and therefore elutes out at a higher percentage of acetonitrile in the HPLC gradient. Mass spectrometry analysis (ESI-MS or MALDI-TOF) is used to confirm the mass of the expected analog (Table 5). NMR analysis of analogs, OCF-S3, and OCF-S4, has been performed to confirm the structure of the analog as a modification on ASP7 (FIGS. 3A-3B and FIGS. 4A-4B). EDC condensation has also been performed on OCF-A and OCF-B, where a carboxylic acid is not present, with no resulting analogs formed, supporting the specificity of the chemistry.

Minimum inhibitory concentration (MIC) assays were performed according to the CLSI M27-A3 method against three indicator strains; S. cerevisiae DGY6, C. albicans ATCC3147, and C. glabrata ATCC2001. ASP7 occidiofungin, OCF-I and OCF-J, have MIC values of 1-2 μg/ml and more active semi-synthetic analogs can be made from OCF-I and OCF-J.

OCF-S3 and OCF-S4, that were synthesized with methylamine used in the EDC condensation reaction, had MIC values which are 2-fold lower compared to OCF-I and OCF-J. An equivalent MIC is seen with OCF-S9 and OCF-S10 which uses propylamine in the EDC condensation. Interestingly, EDC condensation with ethylamine has MIC values of 1-2 μg/ml which is equivalent to OCF-I and OCF-J. A linear relationship would be expected for the changes in antifungal bioactivity observed between the size of the amine used. However, the MIC from methylamine to propylamine do not display this pattern, with OCF-S7 and OCF-S8 (ethylamine) displaying a 4-8-fold increase in MIC compared to OCF-S9 and OCF-S10 (propylamine). Larger primary amines, dodecylamine (MW=185.35 g/mol) and undecylamine (MW=171.32 g/mol), were successful in producing analogs OCF-S11/OCF-S12 and OCF-S13/OCF-S14. OCF-S13 and OCF-S14 had an MIC value of 8 μg/ml against C. glabrata, signifying that the modification region is agreeable to a large structural repertoire of modifications that still enable low micromolar inhibitory activity (Table 6).

EDC mediated condensation of OCF-I and OCF-J with ammonia affords OCF-A and OCF-B variants (named OCF-S1 and OCF-S2), by converting ASP7 to ASN7 (Table 6). The reaction mixture of OCF-S1 and OCF-S2 showed a 2-fold improvement in inhibitory activity compared to OCF-I and OCF-J, but the activity was not restored to the activity reported for OCF-A and OCF-B natural variants (Table 6). Observation of ESI-MS data from the ammonia reaction restored OCF-A and OCF-B variants, but an additional product was also being made with a mass of 1183.56 Da and 1998.57 Da (OCF-S15 and OCF-S16). The mass of these variants in the reaction correspond to an unwanted dehydration side-product of OCF-I and OCF-J named OCF-S15 and OCF-S16. These dehydrated side products have increased MIC values compared to OCF-I and OCF-J, which would result in the disparity in the MIC values reported for the reaction mixture containing OCF-S1, OCF-S2, OCF-S15 and OCF-S16 (Table 6).

TABLE 6
MIC of native occidiofungin variants and semi-synthetic
occidiofungin analogs against S. cerevisiae DGY6, C. albicans
ATCC3147 and C. glabrata ATCC2001 in plain YPD growth media.
OCF-I/OCF-J has greater MIC values compared to OCF-S1/OCF-S2,
OCF-S3/OCF-S4, OCF-S5/OCF-S6, OCF-S9/OCF-S10, and OCF-S19/
OCF-S20. OCF-I/OCF-J has MIC value equal to OCF-S7/OCF-S8
and lower than OCF-S11/OCF-S12, OCF-S13/OCF-S14 and OCF-S21/
OCF-S22 for S. cerevisiae DGY6.

| | Minimum inhibitory concentration | | |
|---|---|---|---|
| | (μg/ml) | | |
| | S. | C. | C. |
| Occidiofungin | cerevisiae | albicans | glabrata |
| variant | DGY6 | ATCC3147 | ATCC2001 |
| OCF-I and OCF-J | 1 | 2 | 2 |
| OCF-S1and OCF-S2* | 0.5 | 1 | 1 |
| OCF-S3 and OCF-S4 | 0.25 | 0.5 | 0.5 |
| OCF-S5 and OCF-S6 | 0.5 | 1 | 0.5 |
| OCF-S7 and OCF-S8 | 1 | 2 | 2 |
| OCF-S9 and OCF-S10 | 0.25 | 0.5 | 0.5 |
| OCF-S11 and OCF-S12 | <8 | <8 | <8 |
| OCF-S13 and OCF- S14 | <8 | <8 | 8 |
| OCF-S15 and OCF- S16 | 2 | 4 | 4 |
| OCF-S19 and OCF-S20 | 0.5 | 1 | 1 |
| OCF-S21 and OCF-S22 | 16 | ND | ND |
| OCF-S21 and OCF-S22 | 4 | ND | ND |
| supplemented with alkaline | | | |
| phosphatase | | | |
| OCF-S25 and OCF-S26 | 0.25 | 0.5 | 0.5 |

ND means not determined.
*MIC for OCF-S1 and OCF-S2 was determined using a sample containing a mixture of OCF-S1/OCF-S2 and OCF-S15/OCF-S16 as they could not be separated chromatographically after the EDC reaction with OCF-I and OCF-J with ammonia as the amine.

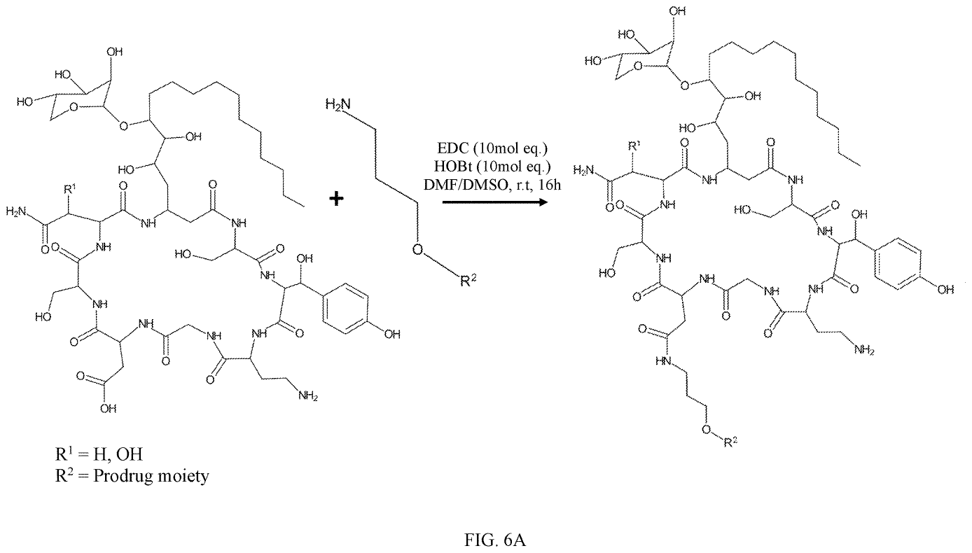

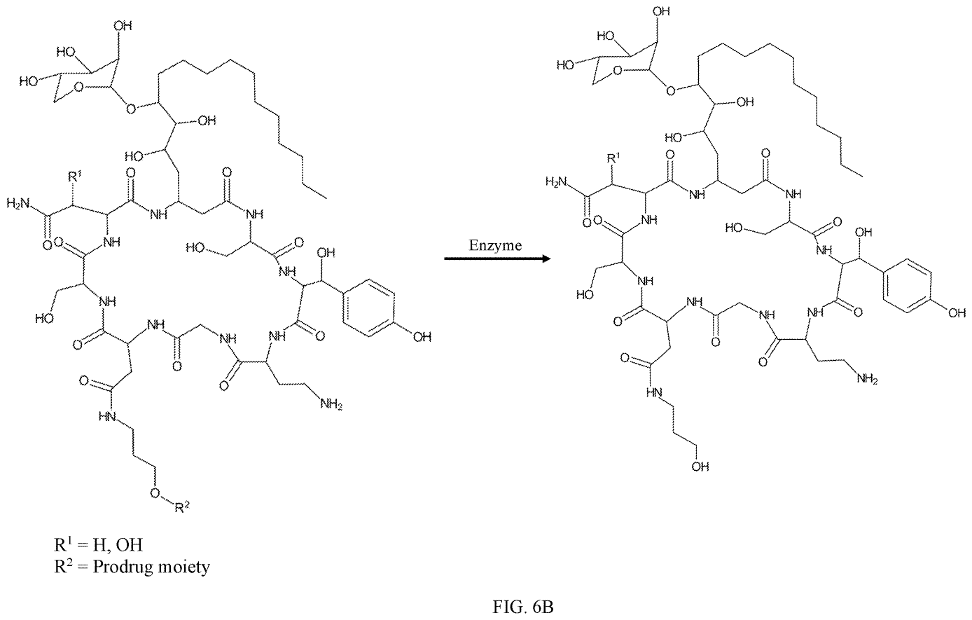

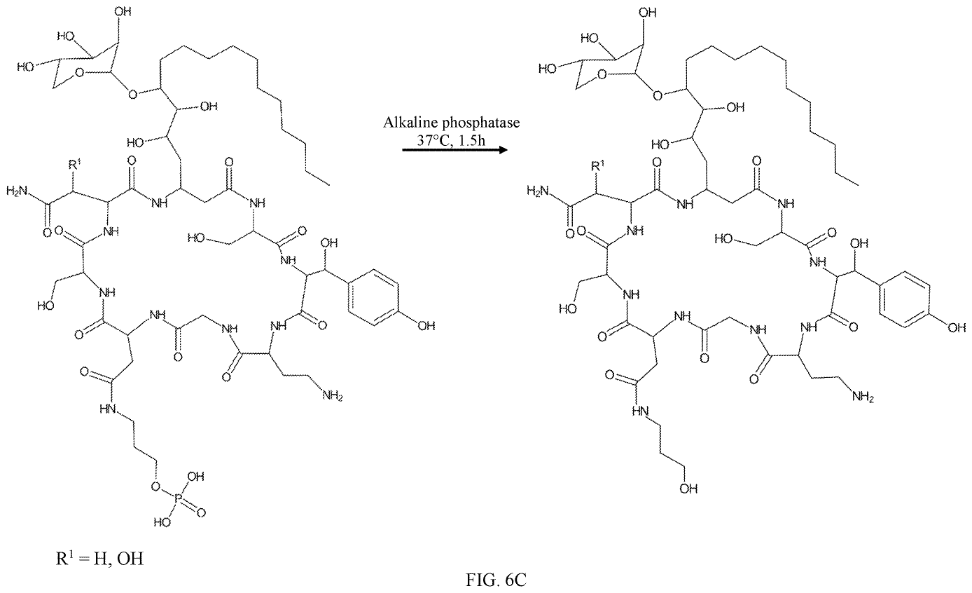

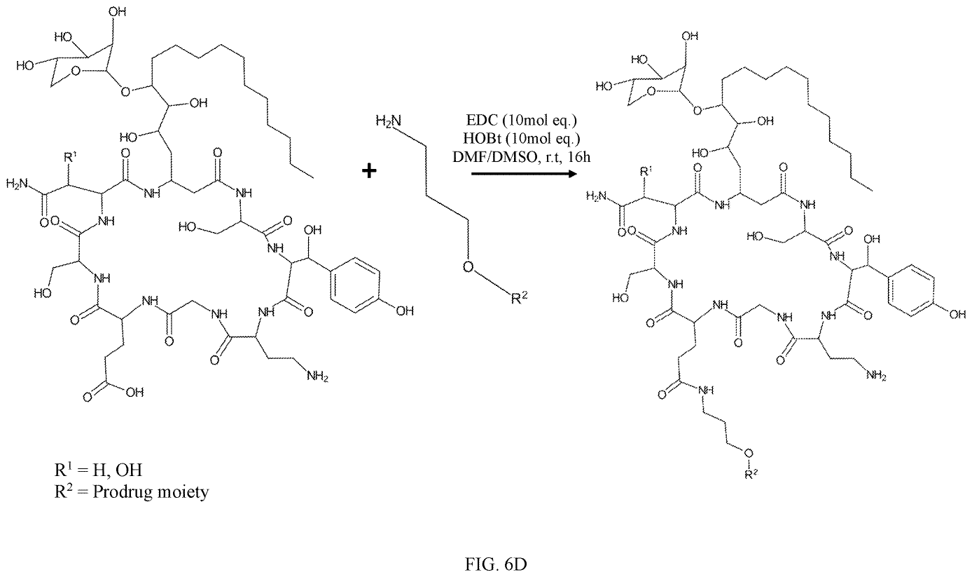

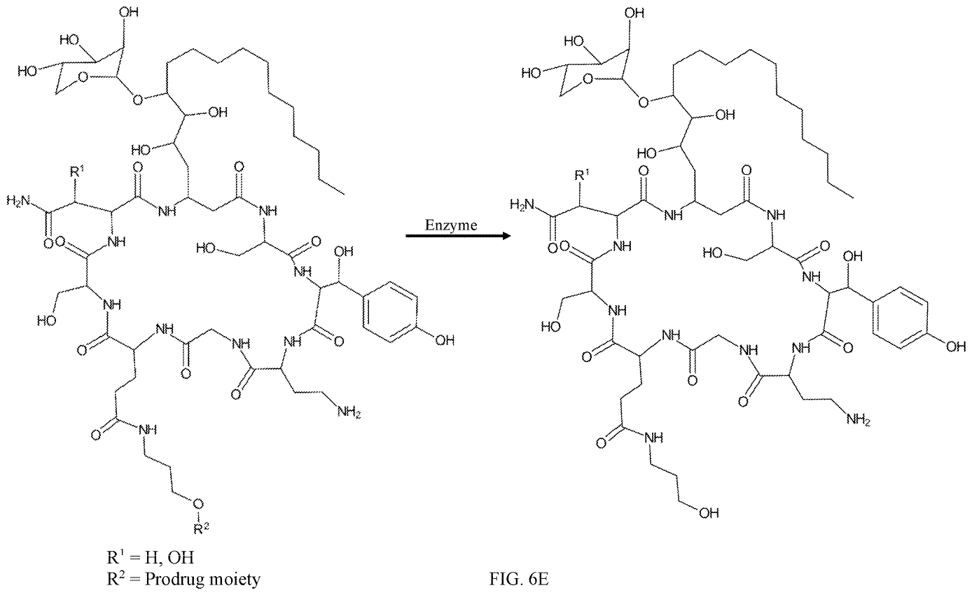

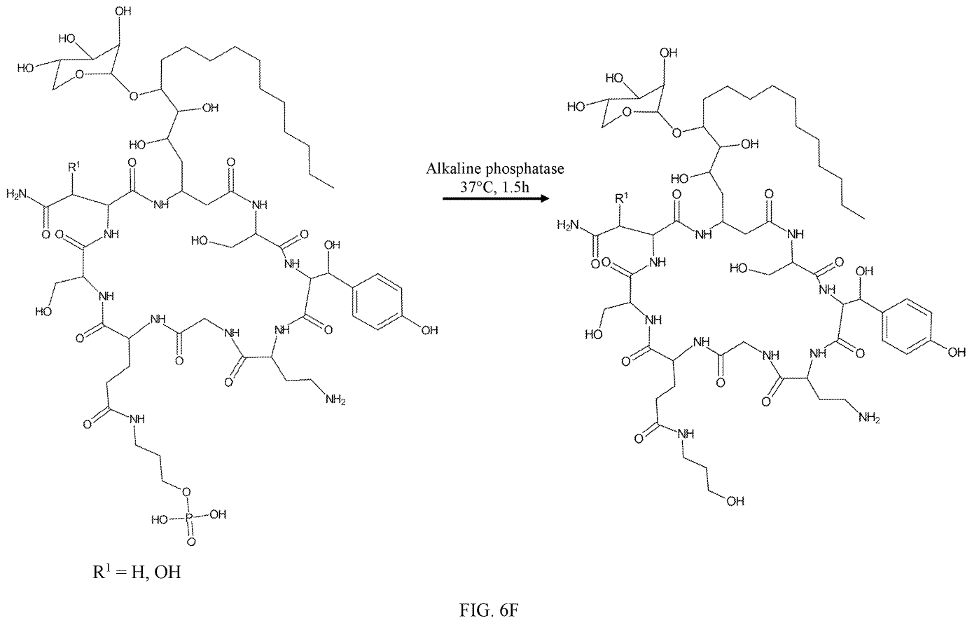

EDC condensation on OCF-I and OCF-J variants can also be performed with amines containing terminal functional groups for the downstream synthesis of various analogs. 3-amino-1-propanol was chosen to produce an analog containing a terminal alcohol group on the ASP7 residue (OCF-S19 and OCF-S20). Primary amines as large as dodecylamine were successful in condensation suggesting that the size of the amine is not interfering with the reaction. The alcohol group of 3-amino-1-propanol was protected by reacting it with tert-butyldimethylsilyl (TBS) chloride and the protected compound was successfully used in EDC condensation to produce OCF-S19 and OCF-S20. These findings suggest that analogs of 3-amino-1-propanol can also participate in EDC condensation with OCF-I and OCF-J (FIG. 6A). The MIC of OCF-S19/OCF-S20 were tested and showed a 2-fold decrease in MIC compared to OCF-I and OCF-J (Table 6). The analog shows improved activity compared to ASP7 occidiofungin and maintains sub-micromolar activity making it a viable linker for the synthesis of a metabolized prodrug compound (FIG. 6B).

An MIC assay with the supplementation of human serum can be an indicator of the effectiveness of a compound in systemic infections and decreased activity is common in drugs tested in the presence of serum due to plasma binding proteins. Semi-synthetic analogs produced using EDC condensation with primary amines showed a 2-8 fold increase in MIC values with serum supplementation (Table 7). OCF-S19 and OCF-S20 showed a 4-8 fold increase in MIC values with serum supplementation (Table 7). These values are comparable to the increase in MIC values of OCF-A and OCF-B in serum.

TABLE 7
MIC of the occidiofungin variants against analogs against S. cerevisiae
DGY6, C. albicans ATCC3147 and C. glabrata ATCC2001 in YPD
growth media supplemented with. 20% human serum. Bracketed numbers
show the fold-increase in comparison to MIC values in plain YPD
media. Occidiofungin semi-synthetic analogs showed 2-8-fold increase
in MIC values in media supplemented in serum.

Minimum inhibitory concentration in
20% human serum (μg/ml)

| Occidiofungin | S. cerevisiae | C. albicans | C. glabrata |
|---|---|---|---|
| variant | DGY6 | ATCC3147 | ATCC2001 |
| OCF-S3 and | 0.5 [x2] | 4 [x8] | 2 [x8] |
| OCF-S4 | | | |
| OCF-S5 and | 1 [x2] | 4 [x4] | 2 [x4] |
| OCF-S6 | | | |
| OCF-S9 and | 1 [x4] | >4 [>x8] | 2 [x4] |
| OCF-S10 | | | |
| OCF-S19 and | 4[x8] | 4[x4] | 8[x8] |
| OCFS-20 | | | |

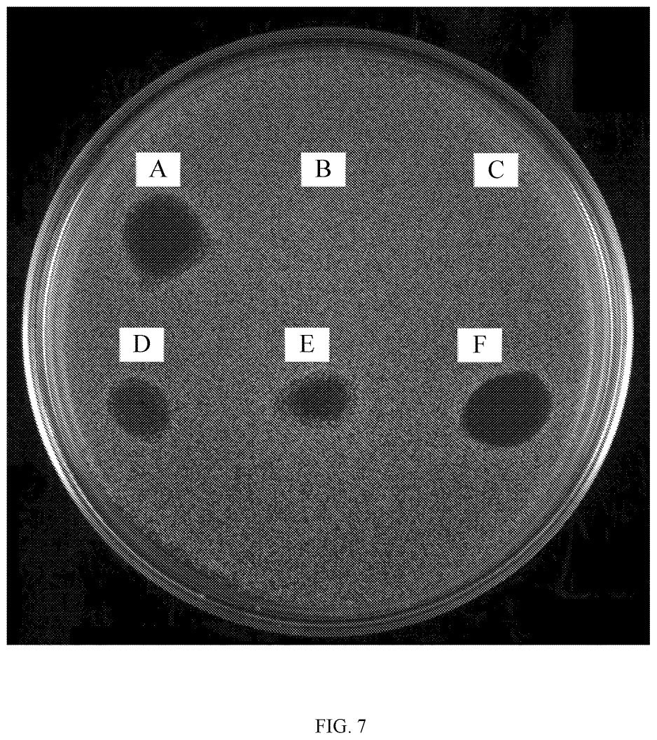

OCF-S19 and OCF-S20 were chosen to be the final metabolized product of a prodrug analog since potent antifungal activity is observed in vitro. The terminal alcohol group would act as a linker arm to a prodrug functional group that can be metabolized to result in the product release of OCF-S19 and OCF-S20 (FIG. 6B). Prodrug functional groups that have been investigated are a phosphate group, methyl succinate and benzoic acid ester. The current lead design is the phosphate prodrug analog OCF-S21 and OCF-S22 where a phosphatase enzyme removes the phosphate group to produce OCF-S19 and OCF-S20 (FIG. 6C). The removal of the phosphate group from OCF-S21 and OCF-S22 to produce OCF-S19 and OCF-S20 was confirmed by MALDI-MS. Fosfluconazole is an example of a phosphate antifungal prodrug which is metabolized by nonselective host alkaline phosphatases into fluconazole^{17,18} Another phosphate prodrug antifungal is fosmanogepix which has recently passed phase 2 clinical trials for the treatment of candidemia¹⁹. OCF-S21 and OCF-S22 had an MIC of 16 μg/ml, which is 16-fold higher compared to OCF-I and OCF-J. The large increase in MIC value is expected due to the addition of the phosphate group. Non-specific alkaline phosphatase (Quick CIP; New England Biolabs) was used to remove the phosphate group from OCF-S21 and OCF-S22 to produce OCF-S19 and OCFS20 which was confirmed by MALDI-TOF MS. An overlay assay with S. cerevisiae DGY6 was used to determine the activity of the prodrug (FIG. 7). The inhibition zone of OCF-S19 and OCF-S20 (FIG. 7; Spot A) was used as a positive control. OCF-S21 and OCF-S22 (FIG. 7; Spot D) had a smaller zone of inhibition in comparison to OCF-S19 and OCF-S20. OCF-S21 and OCF-S22 with alkaline phosphatase added then spotted directly on the plate had no increase in inhibition zone, since the enzyme has not removed the phosphate group yet (FIG. 7; Spot E). Incubating the enzyme with OCF-S21 and OCF-S22 for 90 minutes at 37° C. resulted in inhibition zones sizes comparable to OCF-S19 and OCF-S20 (FIG. 7; Spot F). Alkaline phosphatase reaction mixture spotted directly onto the plate and post incubation had no inhibitory effect on fungal growth (FIG. 7; Spots B and C). Incubating OCF-S21 and OCF-S22 with the enzyme resulted in a 4-fold decrease in MIC values compared to just OCF-S21 and OCF-S22 (Table 6). The reduction in MIC value indicates the removal of the phosphate by the enzyme for antifungal activity. The MIC value of the OCF-S21 and OCF-S22 supplemented with the alkaline phosphatase is not at the same level as OCF-S19 and OCF-S20 which may be a limitation due to components in the media or unoptimized assay conditions. The data supports the premise that alkaline phosphatase found in blood would activate the prodrug over time and support systemic formulations of the prodrug to treat systemic infections or cancer.

Phosphorylated compounds may facilitate activity and pharmacokinetic activity in the presence of serum. In vitro activity in mouse serum is important since any prodrug will first have to be tested in a rodent model, or other small animal models. The alkaline phosphatase used in the activation of OCF-S21 and OCF-S22 into OCF-S19 and OCF-S20 is noted to be non-specific. This highlights that dephosphorylation of OCF-S21 and OCF-S22 is facilitated by a variety of non-specific phosphatases.

Additional prodrugs have been designed with a methyl succinate and benzyl succinate moiety conjugated to the terminal alcohol group of OCF-S19 and OCF-S20. To produce the methyl and methyl succinate prodrugs, 3amino-1-propanol was first conjugated to the methyl or benzyl succinate. N,N′-Dicyclohexylcarbodiimide (DCC) and 4-dimethylaminopyridine (DMAP) was used to conjugate tert-butoxycarbonyl (Boc) 3-amino-1-propanol with the methyl succinate or benzyl succinate, followed by removal of the Boc group. The purified, intermediate compound was then used in EDC condensation to produce the prodrugs of occidiofungin. The modification of the aspartic acid 7 or glutamic acid 7 position, to date position 7 of occidiofungin is the most agreeable point for generating novel analogs of the parent molecule. The EDC mediated condensation reaction provides an effective way to generate a large library of semi-synthetic analogs. EDC condensation can also be used to generate a diverse variety of linkers and functional groups, allowing for a wide variety of analogs to be produced. Further expansion and screening of analogs may lead to the discovery of an analog or analogs that will be active in serum or in other body matrices.

The discovery of OCF-I, OCF-J, and OCF-M and OCF-N presents an amicable target for analog production. Analogs can be selectively produced in an efficient, simple, single-step reaction without the need for protection and deprotection. Furthermore, modifications in the terminal side-chain group of this residue do not have a significant impact on antifungal activity (Table 6).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

### REFERENCES

1. Lu, S. E. et al. Occidiofungin, a unique antifungal glycopeptide produced by a strain of Burkholderia contaminans. Biochemistry 48, 8312-8321 (2009).
2. Ravichandran, A. et al. A novel actin binding drug with in vivo efficacy. Antimicrob. Agents Chemother. 63, 1-13 (2019).
3. Ravichandran, A. et al. Formulation, Pharmacological Evaluation, and Efficacy Studies of Occidiofungin, a Novel Antifungal. Antimicrob. Agents Chemother. 64, (2020).
4. Hing, S. L. et al. Toxicological Evaluation of Occidiofungin against Mice and Human Cancer Cell Lines. Pharmacol. &amp; Pharm. 05, 1085-1093 (2014).
5. Ma, J. et al. Novel antiparasitic activity of the antifungal lead occidiofungin. Antimicrob. Agents Chemother. 64, (2020).
6. Gu, G., Smith, L., Liu, A. & Lu, S. E. Genetic and biochemical map for the biosynthesis of occidiofungin, an antifungal produced by Burkholderia contaminans strain MS14. Appl. Environ. Microbiol. 77, 6189-6198 (2011).
7. Ravichandran, A., Gu, G., Escano, J., Lu, S. E. & Smith, L. The presence of two cyclase thioesterases expands the conformational freedom of the cyclic peptide occidiofungin. J. Nat. Prod. 76, 150-156 (2013).
8. Wüthrich, K. NMR of Proteins and Nucleic Acids. 336-361 (1986) doi:10.1039/9781847555236-00336.
9. F, D. et al. NMRPipe: a multidimensional spectral processing system based on UNIX pipes. J. Biomol. NMR 6, 277-293 (1995).
10. Johnsona, B. A. & Blevinsb, R. A. NMR View: A computer program for the visualization and analysis of NMR data *. J. Biomol. NMR 4, (1994).
11. Izoré, T. et al. Structures of a non-ribosomal peptide synthetase condensation domain suggest the basis of substrate selectivity. Nat. Commun. 2021 121 12, 1-14 (2021).
12. Calcott, M. J., Owen, J. G. & Ackerley, D. F. Efficient rational modification of non-ribosomal peptides by adenylation domain substitution. Nat. Commun. 2020 111 11, 1-10 (2020).
13. Tavares, M. et al. Adaptation and Survival of Burkholderia cepacia and B. contaminans During Long-Term Incubation in Saline Solutions Containing Benzalkonium Chloride. Front. Bioeng. Biotechnol. 8, 630 (2020).
14. Sass, A. M. et al. The unexpected discovery of a novel low-oxygen-activated locus for the anoxic persistence of Burkholderia cenocepacia. ISME J. 7, 1568 (2013).
15. Morgan, G. L. et al. Specificity of Nonribosomal Peptide Synthetases in the Biosynthesis of the Pseudomonas virulence factor. Biochemistry 58, 5249-5254 (2019).
16. Ravichandran, A., Gu, G., Escano, J., Lu, S.-E. & Smith, L. The Presence of Two Cyclase Thioesterases Expands the Conformational Freedom of the Cyclic Peptide Occidiofungin. J. Nat. Prod. 76, 150 (2013).
17. Bentley, A. et al. The discovery and process development of a commercial route to the water soluble prodrug, fosfluconazole. Org. Process Res. Dev. 6, (2002).
18. Aoyama, T. et al. Population pharmacokinetics of fluconazole after administration of fosfluconazole and fluconazole in critically ill patients. J. Clin. Pharm. Ther. 37, (2012).
19. Pappas, P. et al. 147. Clinical Safety and Efficacy of Novel Antifungal, Fosmanogepix, in the Treatment of Candidemia: Results from a Phase 2 Proof of Concept Trial. Open Forum Infect. Dis. 7, (2020).

1-26. (canceled)

27. An analog of an occidiofungin, the occidiofungin analog having Formula (I), Formula (II), Formula (III), or Formula (IV): 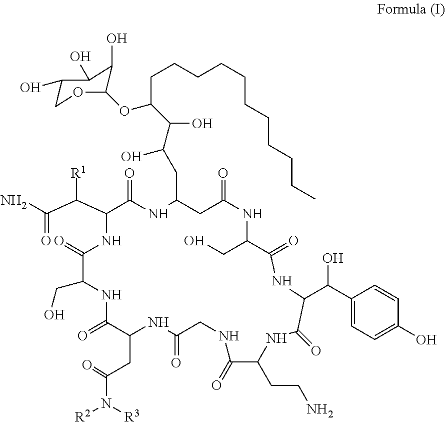
wherein R¹ is H or OH, R² is H or CH₃, and R³ is H, CH₃, CH₂CH₃, CH₂CH₂CH₃, (CH₂)₁₀CH₃, or (CH₂)₁₁CH₃, a terminal alkyne group, a terminal alcohol group, a terminal alcohol group linked to a prodrug functional group, a prodrug functional group, or antibody; 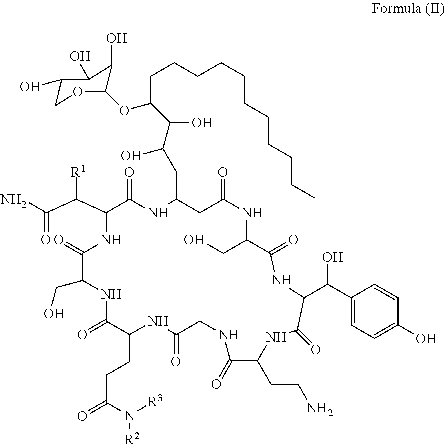
wherein R¹ is H or OH, R² is H or CH₃, and R³ is H, CH₃, CH₂CH₃, CH₂CH₂CH₃, (CH₂)₁₀CH₃, or (CH₂)₁₁CH₃, a terminal alkyne group, a terminal alcohol group, a terminal alcohol group linked to a prodrug functional group, a prodrug functional group, or antibody; 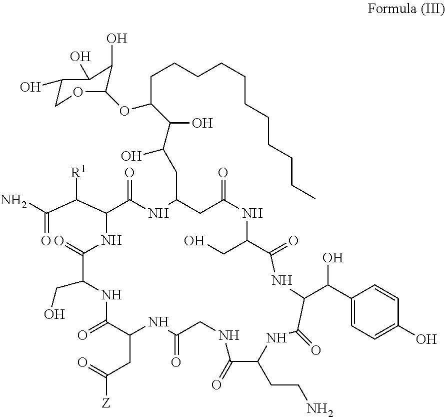
wherein R¹ is H or OH, and Z is an ester, a thioester, an acid anhydride, an acid chloride, an amide or a carboxylate ion; 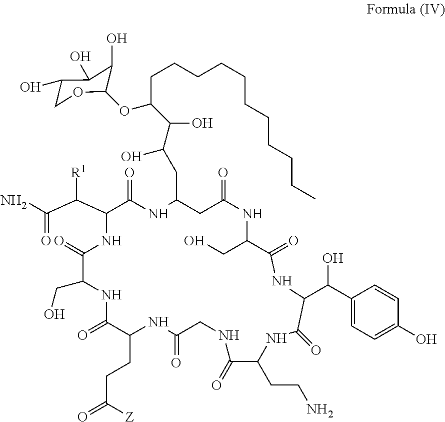
wherein R¹ is H or OH, and Z is an ester, a thioester, an acid anhydride, an acid chloride, an amide or a carboxylate ion.

28. The analog of an occidiofungin of claim 27, wherein the occidiofungin analog is according to Formula (I) or Formula (II): 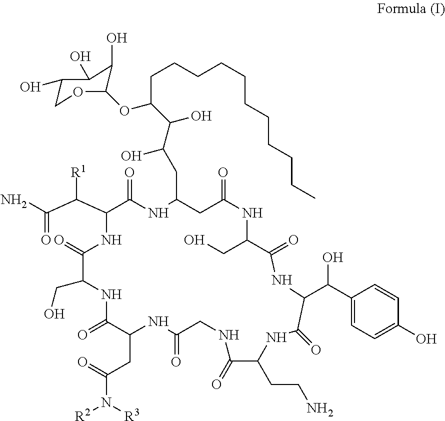
wherein R¹ is H or OH, R² is H, and R³ is H, CH₃, CH₂CH₃, CH₂CH₂CH₃, (CH₂)₁₀CH₃, (CH₂)₁₁CH₃ or CH₂CH₂ CCH; or wherein R¹ is H or OH, R² is CH₃, and R³ is CH₃; 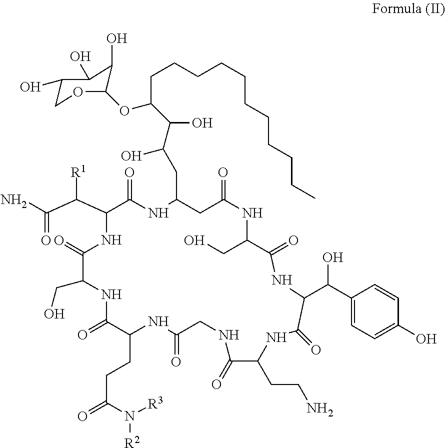
wherein R¹ is H or OH, R² is H, and R³ is H, CH₃, CH₂CH₃, CH₂CH₂CH₃, (CH₂)₁₀CH₃, (CH₂)₁₁CH₃ or CH₂CH₂ CCH; or wherein R¹ is H or OH, R² is CH₃, and R³ is CH₃.

29. The analog of an occidiofungin of claim 27, the occidiofungin analog having Formula (V) or Formula (VI): 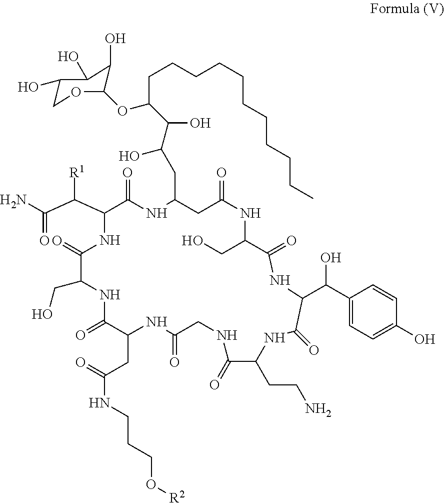
wherein R¹ is H or OH and R² is a prodrug-functional group; 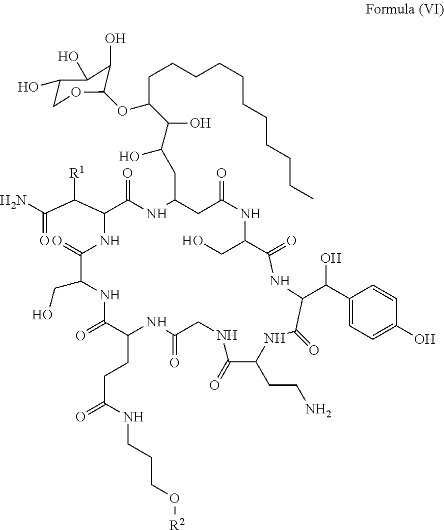
wherein R¹ is H or OH and R² is a prodrug-functional group.

30. The analog of an occidiofungin of claim 29, wherein the prodrug functional group is a phosphate group, methyl succinate, an ether, or benzoic acid ester.

31. The analog of an occidiofungin of claim 29, wherein the analog of occidiofungin is according to Formula (VII) or Formula (VIII): 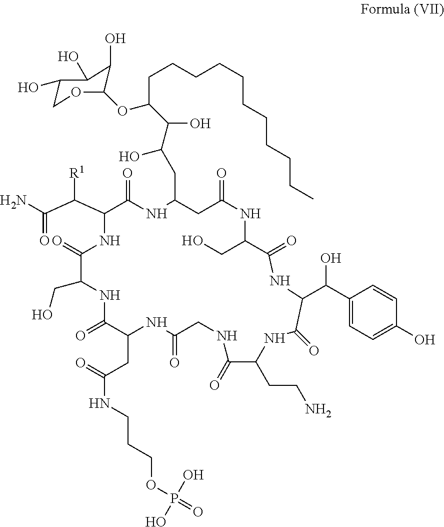
wherein R¹ is H or OH; 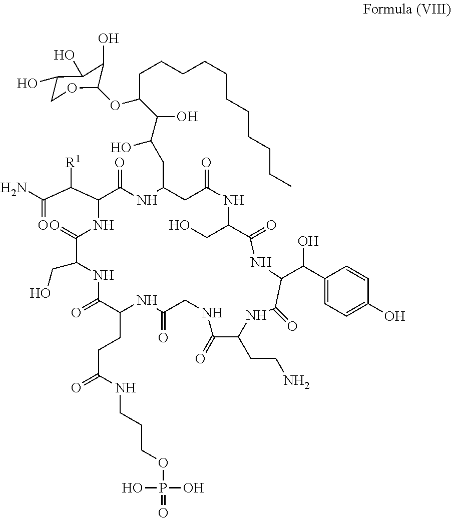
wherein R¹ is H or OH.

32. A pharmaceutical composition comprising an occidiofungin analog of claim 27 and a pharmaceutically acceptable vehicle.

33. A method of treating or preventing a fungal infection, comprising administering to a subject in need thereof an effective amount of an occidiofungin analog of claim 27 or a pharmaceutical composition comprising said analog.

34. The method of claim 33, comprising administering the occidiofungin analog or the pharmaceutical composition orally, topically, intravaginally, intramuscularly, subcutaneously, intrathecally, intravenously or intraperitoneally.

35. The method of claim 33, wherein the fungal infection is caused by a Candida spp., Saccharomyces spp., Trichophyton spp., Rhizopus spp., Mucor spp., Fusarium spp., Aspergillus spp, or Cryptococcus spp.

36. The method of claim 35, wherein the Candida spp. is C. albicans, C. glabrata, C. krusei, C. auris, C. parapsilosis, or C. tropicalis.

37. A method of treating or preventing a parasitic infection, comprising administering to a subject in need thereof an effective amount of an occidiofungin analog of claim 27 or an effective amount of a pharmaceutical composition comprising said analog.

38. A method of treating or preventing cancer, comprising administering to a subject in need thereof an effective amount of an occidiofungin analog of claim 27 or an effective amount of a pharmaceutical composition comprising said analog.

39. A method of producing an occidiofungin analog of Formula (III) or Formula (IV), comprising a condensation of an occidiofungin analog of Formula (IX) or Formula (X), respectively, mediated by 1-ethyl-3-(3-dimethylaminopropyl) carbodiimide hydrochloride (EDC) with an amine: 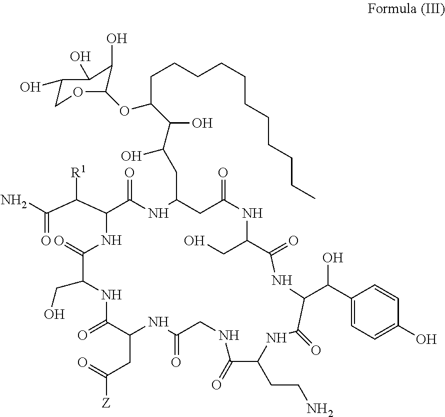
wherein R¹ is H or OH, and Z is an ester, a thioester, an acid anhydride, an acid chloride, an amide or a carboxylate ion; 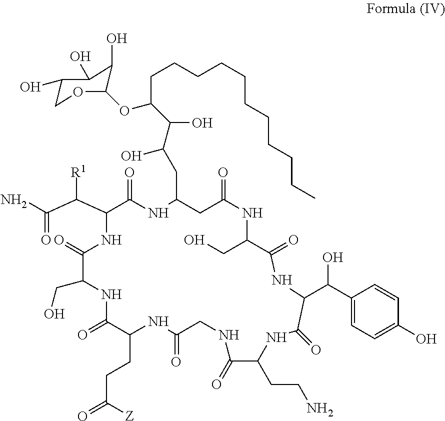
wherein R¹ is H or OH, and Z is an ester, a thioester, an acid anhydride, an acid chloride, an amide or a carboxylate ion; 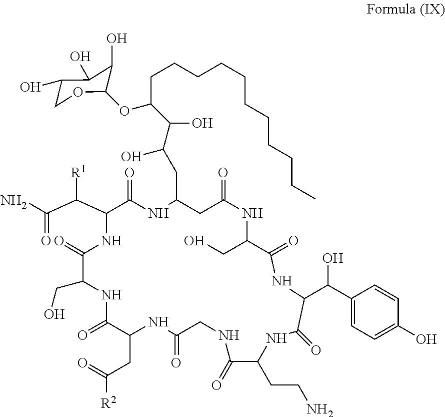
wherein R¹ is H or OH and R² is NH₂ or OH; 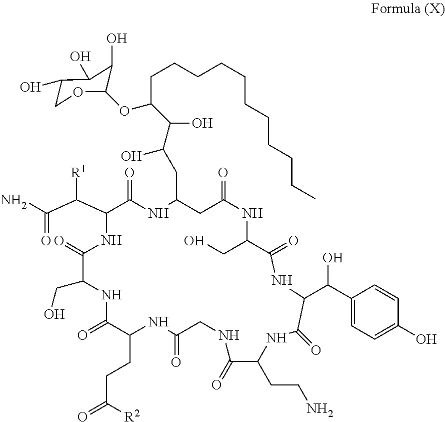
wherein R¹ is H or OH and R² is NH₂ or OH.

40. The method of claim 39, wherein the amine is ammonia, methylamine, dimethylamine, ethylamine, propylamine, undecylamine, dodecylamine, but-3-yn-1-amine, 3-amino-1-propanol, protected 3-amino-1-propanol, 3-amino-1-propanol conjugated to methyl succinate, 3-amino-1-propanol conjugated to benzyl succinate, or 3-amino-1-propanol conjugated to a phosphate group.

41. The method of claim 39, further comprising adding 1-hydroxybenzo-triazole (HOBt) to the occidiofungin analog of Formula III, EDC, and the amine.

42. The method of claim 39, wherein the condensation reaction is carried out in N,N-Dimethylformamide or dimethyl sulfoxide.

43. A method of producing an occidiofungin analog of Formula (V) or Formula (VI), comprising a condensation of an occidiofungin analog of Formula (IX) or Formula (X), respectively, mediated by EDC with an amine containing a prodrug functional group to produce an terminal alcohol arm containing occidiofungin: 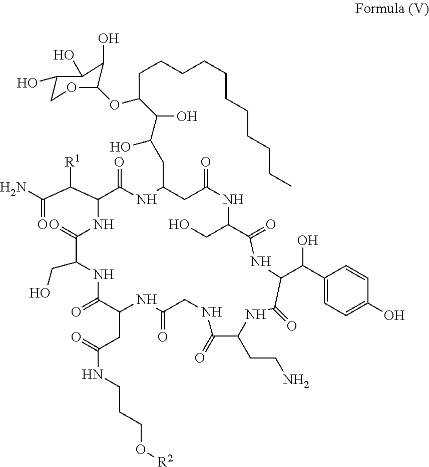
wherein R¹ is H or OH and R² is a prodrug-functional group; 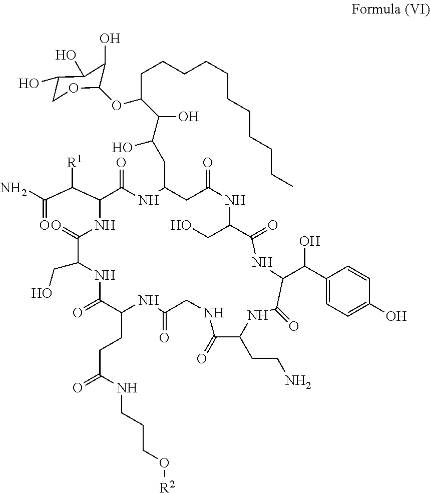
wherein R¹ is H or OH and R² is a prodrug-functional group; 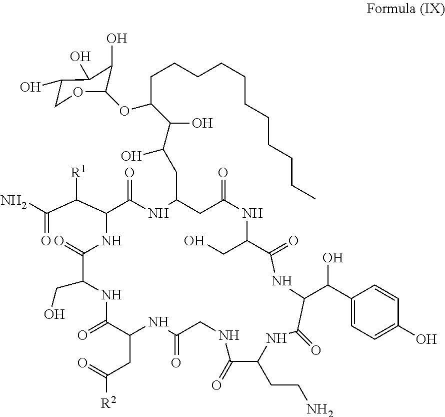
wherein R¹ is H or OH and R² is NH₂ or OH; 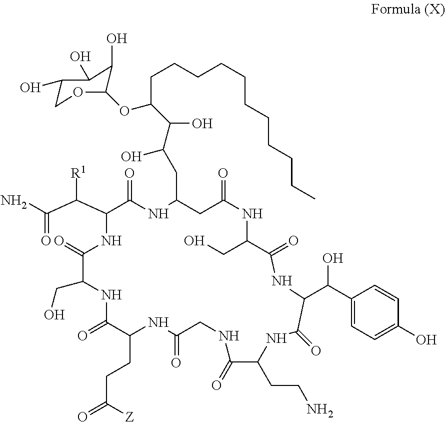
wherein R¹ is H or OH and R² is NH₂ or OH.

44. The method of claim 43, wherein the prodrug functional group is a phosphate group, methyl succinate, or benzyl succinate.

45. The method of claim 43, wherein the amine is 3-amino-1-propanol, protected 3-amino-1-propanol, 3-amino-1-propanol conjugated to methyl succinate, or 3-amino-1-propanol conjugated to benzyl succinate.

46. The method of claim 43, further comprising adding 1-hydroxybenzo-triazole (HOBt) to the occidiofungin analog of Formula III, 1-ethyl-3-(3-dimethylaminopropyl)carbodiimide hydrochloride (EDC), and the amine containing the prodrug functional group.


SEQUENCE LISTING (14 sequences)

SEQ ID NO: 1 - protein, 8 aa, Burkholderia contaminans
  SITE 2: note = MISC_FEATURE - Xaa = 3 amino-5,6-dihydroxy-7-O-xylose-octadecanoic acid
  SITE 4: note = MISC_FEATURE - Xaa = beta-hydroxy tyrosine
  SITE 5: note = MISC_FEATURE - Xaa = 2,4-diaminobutyric acid
NXSXXGNS

SEQ ID NO: 2 - protein, 8 aa, Burkholderia contaminans
  SITE 1: note = MISC_FEATURE - Xaa = beta-hydroxy asparagine
  SITE 2: note = MISC_FEATURE - Xaa = 3 amino-5,6-dihydroxy-7-O-xylose-octadecanoic acid
  SITE 4: note = MISC_FEATURE - Xaa = beta-hydroxy tyrosine
  SITE 5: note = MISC_FEATURE - Xaa = 2,4-diaminobutyric acid
XXSXXGNS

SEQ ID NO: 3 - protein, 8 aa, Burkholderia contaminans
  SITE 2: note = MISC_FEATURE - Xaa = 3 amino-5,6-dihydroxy-7-O-xylose-octadecanoic acid
  SITE 4: note = MISC_FEATURE - Xaa = chlorinated beta-hydroxy tyrosine
  SITE 5: note = MISC_FEATURE - Xaa = 2,4-diaminobutyric acid
NXSXXGNS

SEQ ID NO: 4 - protein, 8 aa, Burkholderia contaminans
  SITE 1: note = MISC_FEATURE - Xaa = beta-hydroxy asparagine
  SITE 2: note = MISC_FEATURE - Xaa = 3 amino-5,6-dihydroxy-7-O-xylose-octadecanoic acid
  SITE 4: note = MISC_FEATURE - Xaa = chlorinated beta-hydroxy tyrosine
  SITE 5: note = MISC_FEATURE - Xaa = 2,4-diaminobutyric acid
XXSXXGNS

SEQ ID NO: 5 - protein, 8 aa, Burkholderia contaminans
  SITE 2: note = MISC_FEATURE - Xaa = 3 amino-5,6-dihydroxy-7-O-xylose-octadecanoic acid delta xylose
  SITE 4: note = MISC_FEATURE - Xaa = beta-hydroxy tyrosine
  SITE 5: note = MISC_FEATURE - Xaa = 2,4-diaminobutyric acid
NXSXXGNS

SEQ ID NO: 6 - protein, 8 aa, Burkholderia contaminans
  SITE 1: note = MISC_FEATURE - Xaa = beta-hydroxy asparagine
  SITE 2: note = MISC_FEATURE - Xaa = 3 amino-5,6-dihydroxy-7-O-xylose-octadecanoic acid delta xylose
  SITE 4: note = MISC_FEATURE - Xaa = beta-hydroxy tyrosine
  SITE 5: note = MISC_FEATURE - Xaa = 2,4-diaminobutyric acid
XXSXXGNS

SEQ ID NO: 7 - protein, 8 aa, Burkholderia contaminans
  SITE 2: note = MISC_FEATURE - Xaa = 3 amino-5,6-dihydroxy-7-O-xylose-octadecanoic acid delta xylose
  SITE 4: note = MISC_FEATURE - Xaa = beta-hydroxy tyrosine
  SITE 5: note = MISC_FEATURE - Xaa = 2,4-diaminobutyric acid
NXSXXGDS

SEQ ID NO: 8 - protein, 8 aa, Burkholderia contaminans
  SITE 1: note = MISC_FEATURE - Xaa = beta-hydroxy asparagine
  SITE 2: note = MISC_FEATURE - Xaa = 3 amino-5,6-dihydroxy-7-O-xylose-octadecanoic acid delta xylose
  SITE 4: note = MISC_FEATURE - Xaa = beta-hydroxy tyrosine
  SITE 5: note = MISC_FEATURE - Xaa = 2,4-diaminobutyric acid
XXSXXGDS

SEQ ID NO: 9 - protein, 8 aa, Burkholderia contaminans
  SITE 2: note = MISC_FEATURE - Xaa = 3 amino-5,6-dihydroxy-7-O-xylose-octadecanoic acid
  SITE 4: note = MISC_FEATURE - Xaa = beta-hydroxy tyrsone
  SITE 5: note = MISC_FEATURE - Xaa = 2,4-diaminobutyric acid
NXSXXGDS

SEQ ID NO: 10 - protein, 8 aa, Burkholderia contaminans
  SITE 1: note = MISC_FEATURE - Xaa = beta-hydroxy asparagine
  SITE 2: note = MISC_FEATURE - Xaa = 3 amino-5,6-dihydroxy-7-O-xylose-octadecanoic acid
  SITE 4: note = MISC_FEATURE - Xaa = beta-hydroxy tyrosine
  SITE 5: note = MISC_FEATURE - Xaa = 2,4-diaminobutyric acid
XXSXXGDS

SEQ ID NO: 11 - protein, 8 aa, Burkholderia contaminans
  SITE 2: note = MISC_FEATURE - Xaa = 3 amino-5,6-dihydroxy-7-O-xylose-octadecanoic acid
  SITE 4: note = MISC_FEATURE - Xaa = beta-hydroxy tyrosine
  SITE 5: note = MISC_FEATURE - Xaa = 2,4-diaminobutyric acid
NXSXXGQS

SEQ ID NO: 12 - protein, 8 aa, Burkholderia contaminans
  SITE 1: note = MISC_FEATURE - Xaa = beta-hydroxy asparagine
  SITE 2: note = MISC_FEATURE - Xaa = 3 amino-5,6-dihydroxy-7-O-xylose-octadecanoic acid
  SITE 4: note = MISC_FEATURE - Xaa = beta-hydroxy tyrosine
  SITE 5: note = MISC_FEATURE - Xaa = 2,4-diaminobutyric acid
XXSXXGQS

SEQ ID NO: 13 - protein, 8 aa, Burkholderia contaminans
  SITE 2: note = MISC_FEATURE - Xaa = 3 amino-5,6-dihydroxy-7-O-xylose-octadecanoic acid
  SITE 4: note = MISC_FEATURE - Xaa = beta-hydroxy tyrosine
  SITE 5: note = MISC_FEATURE - Xaa = 2,4-diaminobutyric acid
NXSXXGES

SEQ ID NO: 14 - protein, 8 aa, Burkholderia contaminans
  SITE 1: note = MISC_FEATURE - Xaa = beta-hydroxy asparagine
  SITE 2: note = MISC_FEATURE - Xaa = 3 amino-5,6-dihydroxy-7-O-xylose-octadecanoic acid
  SITE 4: note = MISC_FEATURE - Xaa = beta-hydroxy tyrosine
  SITE 5: note = MISC_FEATURE - Xaa = 2,4-diaminobutyric acid
XXSXXGES